(19) United States
(12) Patent Application Publication
(10) Pub. No.: US 2019/0153538 A1
(43) Pub. Date: May 23, 2019

(54) GENE EXPRESSION BIOMARKERS FOR PERSONALIZED CANCER CARE TO EPIGENETIC MODIFYING AGENTS

(71) Applicant: Oryzon Genomics, S.A., Madrid (ES)

(72) Inventors: Wei-Yi CHENG, New York, NY (US); Mark D. DEMARIO, New York, NY (US); Fiona MACK, New York, NY (US); Francesca MILLETTI, New York, NY (US); William E. PIERCEALL, New York, NY (US)

(21) Appl. No.: 15/766,086

(22) PCT Filed: Oct. 8, 2016
(86) PCT No.: PCT/EP2016/073821

§ 371 Date: Apr. 5, 2018

Related U.S. Application Data
(60) Provisional application No. 62/239,496, filed on Oct. 9, 2015.
(60) Provisional application No. 62/260,805, filed on Nov. 30, 2015.

(51) Int. Cl. C12Q 1/6886; G16H 50/30; G16H 50/20

(52) U.S. Cl. CPC C12Q 1/6886; G16H 50/30; C12Q 2600/156; C12Q 2600/106; C12Q 2600/158; G16H 50/20

(57) ABSTRACT

The present application discloses a method to predict responsiveness of a patient, with cancer, to treatment with LSD1 inhibitors, said method comprising measuring mRNA expression levels of one or more genes selected from the list of ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB, BCL2 and MYC.

### FIELD OF THE INVENTION

The present invention relates to a method to predict the responsiveness of a patient with a neoplastic disease to treatment with LSD1 inhibitors, said method comprising measuring mRNA expression levels of one or more genes selected from ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB, BCL2, and MYC.

### BACKGROUND OF THE INVENTION

Aberrant gene expression in affected tissue as compared to normal tissue is a common characteristic of many human diseases. This is true for cancer and many neurological diseases which are characterized by changes in gene expression patterns. Gene expression patterns are controlled at multiple levels in the cell. Control of gene expression can occur through modifications of DNA: DNA promoter methylation is associated with suppression of gene expression. Several inhibitors of DNA methylation are approved for clinical use including the blockbuster Vidaza™. Another class of modifications involve histones which form the protein scaffold that DNA is normally associated with (coiled around) in eukaryotic cells. Histones play a crucial role in organizing DNA and the regulated coiling and uncoiling of DNA around the histones is critical in controlling gene expression—coiled DNA is typically not accessible for gene transcription. A number of histone modifications have been discovered including histone acetylation, histone lysine methylation, histone arginine methylation, histone ubiquinylation, and histone sumoylation, many of which modify accessibility to the associated DNA by the cells transcriptional machinery. These histone marks serve to recruit various protein complexes involved in transcription and repression. An increasing number of studies are painting an intricate picture of how various combinations of histone marks control gene expression in cell-type specific manner and a new term has been coined to capture this concept: the histone code.

The prototypical histone mark is histone acetylation. Histone acetyl transferase and histone deacetylases are the catalytic machines involved in modulation of this histone mark although typically these enzymes are parts of multiprotein complexes containing other proteins involved in reading and modifying histone marks. The components of these protein complexes are typically cell-type specific and typically comprise transcriptional regulators, repressors, co-repressors, receptors associated with gene expression modulation (e.g., estrogen or androgen receptor). Histone deacetylase inhibitors alter the histone acetylation profile of chromatin. Accordingly, histone deacetylase inhibitors like Vorinostat (SAHA), Trichostatin A (TSA), and many others have been shown to alter gene expression in various in vitro and in vivo animal models. Clinically, histone deacetylase inhibitors have demonstrated activity in the cancer setting and are being investigated for oncology indications as well as for neurological conditions and other diseases.

Another modification that is involved in regulating gene expression is histone methylation including lysine and arginine methylation. The methylation status of histone lysines has recently been shown to be important in dynamically regulating gene expression.

A group of enzymes known as histone lysine methyl transferases and histone lysine demethylases are involved in histone lysine modifications. One particular human histone lysine demethylase enzyme called Lysine Specific Demethylase-1 (LSD1) was recently discovered (Shi et al. (2004) Cell 119:941) to be involved in this crucial histone modification. LSD1 has a fair degree of structural similarity, and amino acid identity/homology to polyamine oxidases and monoamine oxidases, all of which (i.e., MAO-A, MAO-B and LSD1) are flavin dependent amine oxidases which catalyze the oxidation of nitrogen-hydrogen bonds and/or nitrogen carbon bonds. LSD1 has been recognized as an interesting target for the development of new drugs to treat cancer, neurological diseases and other conditions.

Cyclopropylamine containing compounds are known to inhibit a number of medically important targets including amine oxidases like Monoamine Oxidase A (MAO-A; or MAOA), Monoamine Oxidase B (MAO-B; or MAOB), and Lysine Specific Demethylase-1 (LSD1). Tranylcypromine (also known as 2-phenylcyclopropylamine), which is the active ingredient of Parnate® and one of the best known examples of a cyclopropylamine, is known to inhibit all of these enzymes. Since MAO-A inhibition may cause undesired side effects, it would be desirable to identify cyclopropylamine derivatives that exhibit potent LSD1 inhibitory activity while being devoid of or having substantially reduced MAO-A inhibitory activity.

Compounds which act as inhibitors of LSD1 are known in the art. LSD1 inhibitors and methods for making them are for example disclosed in WO 2011/131697 (A1), WO 2012135113 (A2), WO 2013/057322 (A1), WO 2010/143582, WO 2011/131576, WO 2013/022047, WO 2013/025805, WO 2014/058071, WO 2014/084298, WO 2014/085613, WO 2014/086790, WO2014/164867, WO 2014/194280, WO 2014/205213, WO 2015/021128, WO 2015/031564, WO 2015/089192, WO 2015/120281, WO 2015/123465, WO 2015/123437, WO 2015/123424, WO 2015/123408, WO 2015/134973, WO 2015/156417 and WO 2015/168466 which are incorporated in their entirety herein.

WO 2012135113 (A2) discloses compounds, for example GSK2879552 [CAS Reg. No. 1401966-69-5], also known as 4-[[4-[[[(1R,2S)-2-phenylcyclopropyl]amino]methyl]-1-piperidinyl]methyl]-benzoic acid (Example 26 on p. 75, Example 29 on p. 81), as selective LSD1 inhibitor.

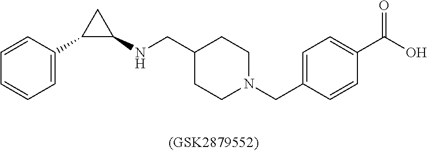 [SMILES: O=C(O)c1ccc(CN2CCC(CN[C@@H]3C[C@H]3c3ccccc3)CC2)cc1]

LSD1 inhibitors and methods for making them are for example disclosed in WO 2011/131697 (A1), particularly examples 1-21 (pages 90 to 103), which are incorporated in their entirety herein.

LSD1 inhibitors and methods for making them are for example disclosed in WO 2013/057322 (A1), particularly examples 1-108 (pages 155 to 191), which are incorporated in their entirety herein.

Particular LSD1 inhibitors described in WO 2013/057322 (A1) are provided in Table 1.

TABLE 1
Particular LSD1 inhibitors disclosed in WO 2013/057322 (A1).

| Example No of | | |
|---|---|---|
| WO 2013/057322 | Substance name | Structure |
| 1 | N1-((trans)-2-phenylcyclopropyl) cyclohexane-1,4-diamine | 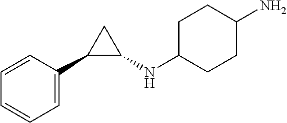 [SMILES: NC1CCC(N[C@H]2C[C@@H]2c2ccccc2)CC1] |
| 5 | (trans)-N1-((1R,2S)-2- phenylcyclopropyl) cyclohexane-1,4-diamine | 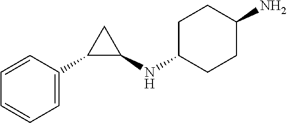 [SMILES: N[C@H]1CC[C@H](N[C@@H]2C[C@H]2c2ccccc2)CC1] |
| 15 | (R)-1-(4-(((trans)-2- phenylcyclopropyl)amino) cyclohexyl)pyrrolidin-3-amine | 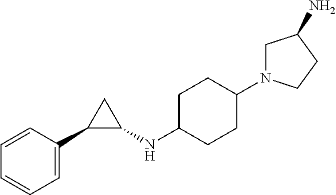 [SMILES: N[C@H]1CCN(C2CCC(N[C@H]3C[C@@H]3c3ccccc3)CC2)C1] |
| 17 | 4-(aminomethyl)-N-((trans)-2- phenylcyclopropyl) cyclohexanamine | 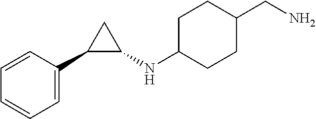 [SMILES: NCC1CCC(N[C@H]2C[C@@H]2c2ccccc2)CC1] |
| 18 | N1-((trans)-2-phenylcyclopropyl) cyclohexane-1,3-diamine | 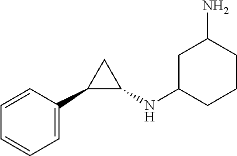 [SMILES: NC1CCCC(N[C@H]2C[C@@H]2c2ccccc2)C1] |
| 19 | N1-((trans)-2-phenylcyclopropyl) cyclobutane-1,3-diamine | 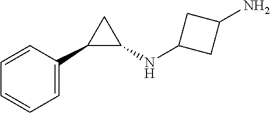 [SMILES: NC1CC(N[C@H]2C[C@@H]2c2ccccc2)C1] |
| 20 | N1-((trans)-2-phenylcyclopropyl)- 2,3-dihydro-1H-indene-1,3-diamine | 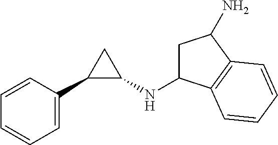 [SMILES: NC1CC(N[C@H]2C[C@@H]2c2ccccc2)c2ccccc21] |
| 22 | N1-methyl-N4-((trans)-2- phenylcyclopropyl) cyclohexane-1,4-diamine | 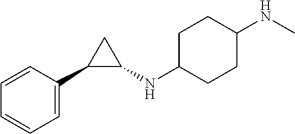 [SMILES: CNC1CCC(N[C@H]2C[C@@H]2c2ccccc2)CC1] |
| 26 | N1-((trans)-2-(4- bromophenyl)cyclopropyl) cyclohexane-1,4-diamine | 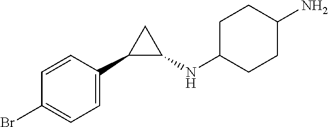 [SMILES: NC1CCC(N[C@H]2C[C@@H]2c2ccc(Br)cc2)CC1] |
| 27 | N1-(2-(o-tolyl)cyclopropyl) cyclohexane-1,4-diamine | 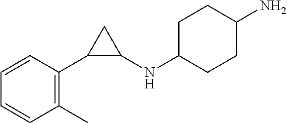 [SMILES: Cc1ccccc1C1CC1NC1CCC(N)CC1] |
| 29 | N1-(2-(4- methoxyphenyl)cyclopropyl) cyclohexane-1,4-diamine | 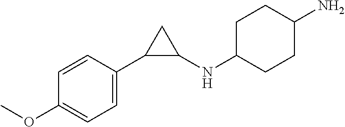 [SMILES: COc1ccc(C2CC2NC2CCC(N)CC2)cc1] |
| 31 | N1-(2-(2-fluorophenyl)cyclopropyl) cyclohexane-1,4-diamine | 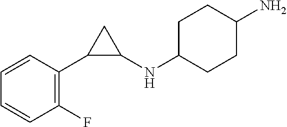 [SMILES: NC1CCC(NC2CC2c2ccccc2F)CC1] |
| 33 | N1-(2-(naphthalen-2- yl)cyclopropyl) cyclohexane-1,4-diamine | 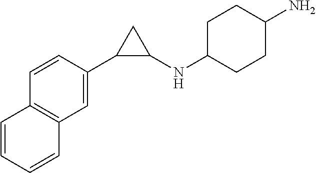 [SMILES: NC1CCC(NC2CC2c2ccc3ccccc3c2)CC1] |
| 50 | N-(4′-((trans)-2-((4- aminocyclohexyl)amino) cyclopropyl)-[1,1′-biphenyl]-3-yl)- 2-cyanobenzenesulfonamide | 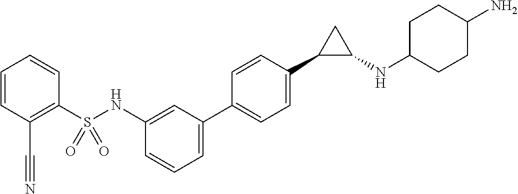 [SMILES: N#Cc1ccccc1S(=O)(=O)Nc1cccc(-c2ccc([C@H]3C[C@@H]3NC3CCC(N)CC3)cc2)c1] |
| 56 | N1-((trans)-2-(4-(pyridin-3- ylmethoxy)phenyl)cyclopropyl) cyclohexane-1,4-diamine | 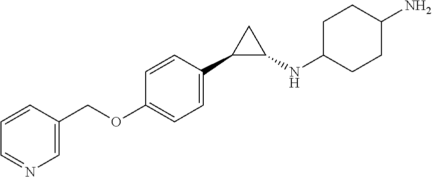 [SMILES: NC1CCC(N[C@H]2C[C@@H]2c2ccc(OCc3cccnc3)cc2)CC1] |

A more particular LSD1 inhibitor described in WO 2013/057322 (A1) is (trans)-N1-((1R,2S)-2-phenylcyclopropyl)cyclohexane-1,4-diamine [CAS Reg. No. 1431304-21-0]

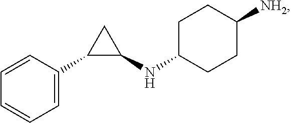 [SMILES: N[C@H]1CC[C@H](N[C@@H]2C[C@H]2c2ccccc2)CC1]

corresponding to Example 5 therein, and pharmaceutically acceptable salts thereof.

Even though potent selective LSD1 inhibitors have been proposed for adequate treatments for conditions such as cancer and neurodegeneration, biomarkers for personalized treatment have not been described.

It has long been acknowledged that there is a need to develop methods of individualizing cancer treatment. With the development of targeted cancer treatments, there is a particular need for prognostic and even more so in predictive markers, i.e. factors predicting differential efficacy of a particular therapy based on marker status (e.g., only patients expressing the marker will or will not benefit from a specific therapeutic regimen).

Therefore, it is an aim of the present invention to provide biomarkers that are predictive for response and outcome to LSD1 inhibitor treatment in patients with neoplastic diseases.

### DETAILED DESCRIPTION OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

All publications, patent applications, patents, and other references mentioned herein are incorporated by reference in their entirety.

The nomenclature used in this Application is based on IUPAC systematic nomenclature, unless indicated otherwise.

Any open valency appearing on a carbon, oxygen, sulfur or nitrogen atom in the structures herein indicates the presence of a hydrogen, unless indicated otherwise.

When indicating the number of substituents, the term “one or more” refers to the range from one substituent to the highest possible number of substitution, i.e. replacement of one hydrogen up to replacement of all hydrogens by substituents.

The term “optional” or “optionally” denotes that a subsequently described event or circumstance can but need not occur, and that the description includes instances where the event or circumstance occurs and instances in which it does not.

“The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511). The prefixes D and L or (+) and (−) are employed to designate the sign of rotation of plane-polarized light by the compound, with (−) or L designating that the compound is levorotatory. A compound prefixed with (+) or D is dextrorotatory.

The terms “pharmaceutical composition” and “pharmaceutical formulation” (or “formulation”) are used interchangeably and denote a mixture or solution comprising a therapeutically effective amount of an active pharmaceutical ingredient together with pharmaceutically acceptable excipients to be administered to a mammal, e.g., a human in need thereof.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “inhibitor” denotes a compound which competes with, reduces or prevents the binding of a particular ligand to a particular receptor or enzyme and/or which reduces or prevents the activity of a particular protein, e.g. of a receptor or an enzyme.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “animal” as used herein comprises human beings and non-human animals. In one embodiment, a “non-human animal” is a mammal, for example a rodent such as rat or a mouse. In one embodiment, a non-human animal is a mouse.

The term “half maximal effective concentration” (EC50) denotes the plasma concentration of a particular compound or molecule required for obtaining 50% of the maximum of a particular effect in vivo.

The term “therapeutically effective amount” (or “effective amount”) denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “treating” or “treatment” of a disease state includes inhibiting the disease state, i.e., arresting the development of the disease state or its clinical symptoms, or relieving the disease state, i.e., causing temporary or permanent regression of the disease state or its clinical symptoms.

The term “assessing a neoplastic disease” is used to indicate that the method according to the present invention will aid a medical professional including, e.g., a physician in assessing whether an individual has a neoplastic disease or is at risk of developing a neoplastic disease. The levels of a gene panel as compared to one or more reference levels indicate whether the individual has a neoplastic disease or whether the individual is at risk of developing a neoplastic disease or prognosing the course of a neoplastic disease. In one embodiment the term assessing a neoplastic disease is used to indicate that the method according to the present invention will aid the medical professional in assessing whether an individual has a neoplastic disease or not. In this embodiment levels of a gene panel as compared to one or more reference levels indicate whether the individual has a neoplastic disease.

The term “assessing a therapy” is used to indicate that the method according to the present invention will aid a medical professional including, e.g., a physician in assessing whether an individual having a neoplastic disease should be treated with an effective amount of an LSD1 inhibitor. Levels of the responder genes above the reference level, and/or levels of the non-responder genes below the reference level indicate that the patient should be treated with an effective amount of an LSD1 inhibitor. In certain embodiments, the term “at the reference level” refers to a level of a gene of the gene panel in the sample from the individual or patient that is essentially identical to the reference level or to a level that differs from the reference level by up to 1%, up to 2%, up to 3%, up to 4%, up to 5%.

In certain embodiments, the term “above the reference level” refers to a level of a gene of the gene panel in the sample from the individual or patient above the reference level or to an overall increase of 5%, 10%, 20%, 25%, 30%, 40%, 50%, 60%, 70%, 80%, 85%, 90%, 95%, 100% or greater, determined by the methods described herein, as compared to the reference level. In certain embodiments, the term increase refers to the increase in a level of a gene of the gene panel in the sample from the individual or patient wherein, the increase is at least about 1.5-, 1.75-, 2-, 3-, 4-, 5-, 6-, 7-, 8-, 9-, 10-, 15-, 20-, 25-, 30-, 40-, 50-, 60-, 70-, 75-, 80-, 90-, or 100-fold higher as compared to the reference level, e.g. predetermined from a reference sample.

In certain embodiments, the term “decrease” or “below” herein to a level of a gene of the gene panel in the sample from the individual or patient below the reference level or to an overall reduction of 5%, 10%, 20%, 25%, 30%, 40%, 50%, 60%, 70%, 80%, 85%, 90%, 95%, 96%, 97%, 98%, 99% or greater, determined by the methods described herein, as compared to the reference level. In certain embodiments, the term decrease refers to a decrease in a level of a gene of the gene panel in the sample from the individual or patient wherein the decreased level is at most about 0.9-, 0.8-, 0.7-, 0.6-, 0.5-, 0.4-, 0.3-, 0.2-, 0.1-, 0.05-, or 0.01-fold of the reference level, e.g. predetermined from a reference sample, or lower.

The term “biomarker” as used herein refers generally to a gene, the expression or presence of which in or on a mammalian tissue or cell can be detected by standard methods (or methods disclosed herein) and which may be predictive, diagnostic and/or prognostic for a mammalian cell's or tissue's sensitivity to treatment regimes based on LSD1 inhibition by e.g. an LSD1 inhibitor such as (trans)-N1-((1R,2S)-2-phenylcyclopropyl)cyclohexane-1,4-diamine bis-hydrochloride. In certain embodiments, the level of such a biomarker is determined to be higher or lower than that observed for a reference sample.

The term “comparing” as used herein refers to comparing the level of the biomarker in the sample from the individual or patient with the reference level of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer assisted. Thus, the comparison may be carried out by a computing device (e.g., of a system disclosed herein). The value of the measured or detected level of the biomarker in the sample from the individual or patient and the reference level can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references which are stored in a database by a computer program. The computer program may further evaluate the result of the comparison, i.e. automatically provide the desired assessment in a suitable output format. For a computer assisted comparison, the value of the determined amount may be compared to values corresponding to suitable references which are stored in a database by a computer program. The computer program may further evaluate the result of the comparison, i.e. automatically provides the desired assessment in a suitable output format.

The term “detecting” a biomarker as used herein refers to methods of detecting the presence of quantity of the biomarker in the sample employing appropriate methods of detection described elsewhere herein.

The term “measuring” the level of a biomarker, as used herein refers to the quantification of the biomarker, e.g. to determining the level of the biomarker in the sample, employing appropriate methods of detection described elsewhere herein.

The term “monitoring the efficacy of a therapy” is used to indicate that a sample is obtained at least once, including serially, from a patient before and/or under therapy with an LSD1 inhibitor and that gene panel levels are measured therein to obtain an indication whether the therapy is efficient or not.

In the monitoring of the efficacy of a therapy the gene panel levels are measured and in one embodiment compared to a reference value for the gene panel, or, in a further embodiment, it is compared to the gene panel levels in a sample obtained from the same patient at an earlier point in time, e.g. while said patient was already under therapy or before start of a therapy in said patient.

A “patient” or “subject” herein is any single human subject eligible for treatment who is experiencing or has experienced one or more signs, symptoms, or other indicators of a neoplastic disease. Intended to be included as a subject are any subjects involved in clinical research trials not showing any clinical sign of disease, or subjects involved in epidemiological studies, or subjects once used as controls. The subject may have been previously treated with an LSD1 inhibitor or another drug, or not so treated. The subject may be naïve to an additional drug(s) being used when the treatment herein is started, i.e., the subject may not have been previously treated with, for example, a therapy other than an LSD1 inhibitor at “baseline” (i.e., at a set point in time before the administration of a first dose of Drug D in the treatment method herein, such as the day of screening the subject before treatment is commenced). Such “naïve” subjects are generally considered to be candidates for treatment with such additional drug(s).

The phrase “providing a diagnosis/assessment” as used herein refers to using the information or data generated relating to the gene panel levels in a sample of a patient to diagnose/assess a neoplastic disease in the patient. The information or data may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level.

The phrase “recommending a treatment” as used herein refers to using the information or data generated relating to the gene panel levels in a sample of a patient to identify the patient as suitably treated or not suitably treated with a therapy. In some embodiment the therapy may comprise an LSD1 inhibitor. In some embodiments the phrase “recommending a treatment/therapy” includes the identification of a patient who requires adaptation of an effective amount of an LSD1 inhibitor being administered. In some embodiments recommending a treatment includes recommending that the amount of an LSD1 inhibitor being administered is adapted. The phrase “recommending a treatment” as used herein also may refer to using the information or data generated for proposing or selecting a therapy comprising an LSD1 inhibitor for a patient identified or selected as more or less likely to respond to the therapy comprising a LSD1 inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level. In some embodiments, the information or data includes an indication that the patient is suitably treated or not suitably treated with a therapy comprising an LSD1 inhibitor.

In certain embodiments, the term “reference level” herein refers to a predetermined value. In this context “level” encompasses the absolute amount, the relative amount or concentration as well as any value or parameter which correlates thereto or can be derived therefrom. As the skilled artisan will appreciate the reference level is predetermined and set to meet routine requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90%, 95% or 98%, respectively. These requirements may also be defined in terms of positive or negative predictive values. Nonetheless, based on the teaching given in the present invention it will always be possible for a skilled artisan to arrive at the reference level meeting those requirements. In one embodiment the reference level is determined in reference samples from healthy individuals. The reference level in one embodiment has been predetermined in reference samples from the disease entity to which the patient belongs. In certain embodiments the reference level can e.g. be set to any percentage between 25% and 75% of the overall distribution of the values in a disease entity investigated. In other embodiments the reference level can e.g. be set to the median, tertiles or quartiles as determined from the overall distribution of the values in reference samples from a disease entity investigated. In one embodiment the reference level is set to the median value as determined from the overall distribution of the values in a disease entity investigated. The reference level may vary depending on various physiological parameters such as age, gender or subpopulation, as well as on the means used for the determination of the gene panel levels referred to herein. In one embodiment, the reference sample is from essentially the same type of cells, tissue, organ or body fluid source as the sample from the individual or patient subjected to the method of the invention, e.g. if according to the invention blood is used as a sample to determine the gene panel levels in the individual, the reference level is also determined in blood or a part thereof.

The phrase “responsive to” in the context of the present invention indicates that a patient suffering from, being suspected to suffer or being prone to suffer from, or diagnosed with a disorder as described herein, shows a response to therapy comprising an LSD1 inhibitor.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, bronchial lavage or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation.

The phrase “selecting a patient” or “identifying a patient” as used herein refers to using the information or data generated relating to the gene panel levels in a sample of a patient to identify or selecting the patient as more likely to benefit or less likely to benefit from a therapy comprising an LSD1 inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level. In some embodiments, the information or data includes an indication that the patient is more likely or less likely to respond to a therapy comprising an LSD1 inhibitor.

The phrase “selecting a therapy” as used herein refers to using the information or data generated relating to the gene panel levels in a sample of a patient to identify or selecting a therapy for a patient. In some embodiment the therapy may comprise an LSD1 inhibitor. In some embodiments the phrase “identifying/selecting a therapy” includes the identification of a patient who requires adaptation of an effective amount of an LSD1 inhibitor being administered. In some embodiments recommending a treatment includes recommending that the amount of LSD1 inhibitor being administered is adapted. The phrase “recommending a treatment” as used herein also may refer to using the information or data generated for proposing or selecting a therapy comprising an LSD1 inhibitor for a patient identified or selected as more or less likely to respond to the therapy comprising an LSD1 inhibitor. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the gene panel levels to a reference level. In some embodiments, the information or data includes an indication that a therapy comprising an LSD1 inhibitor is suitable for the patient.

The term “responder gene” refers to the group of genes comprising ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB and BCL2, particularly to the group of genes comprising ASCL1, DDC, CEACAM6, LRRIQ4, GRP, NROB2, CEACAM5, SOX21, OR51E2, SEC11C, BAALC, CCDC40, RAB3B, RGS17, ABCE1, ETS2, CCDC154, SPAG6, PON1, TMEM176A, C1orf127, IGFBP5, IGF2BP2, FAM84A, FOXA2, HOXA10, or alternatively to the group of genes comprising ASCL1, HOXA10, NCAM1, NCAM2, NEUROD1, DDC, GRP, KRT8, ENO2, AVP, OXT, SYP, CHGA, CHGB, SOX21 and BCL2.

The term “non-responder gene” refers to the oncogene MYC.

Table 2 provides a list including description of the genes employed in present invention.

TABLE 2
Description of the genes employed in the invention (*http://www.ensembl.org/,
Cunningham F. et al., Nucl. Acids Res. (2015) 43(D1): D662-D669).

| Gene | Ensembl Gene ID* | Description | Synonyms | Location: Chromosome |
|---|---|---|---|---|
| ASCL1 | ENSG00000139352 | achaete-scute family | ASH1, bHLHa46, | Chromosome 12: |
| | | bHLH transcription | HASH1 | 102,957,686-102,960,516 |
| | | factor 1 | | forward strand. |
| DDC | ENSG00000132437 | dopa decarboxylase | AADC | Chromosome 7: |
| | | | | 50,458,436-50,565,457 |
| | | | | reverse strand. |
| CEACAM6 | ENSG00000086548 | carcinoembryonic | CD66c, NCA | Chromosome 19: |
| | | antigen-related cell | | 41,750,977-41,772,208 |
| | | adhesion molecule 6 | | forward strand. |
| LRRIQ4 | ENSG00000188306 | leucine-rich repeats | LRRC64 | Chromosome 3: |
| | | and IQ motif | | 169,821,922-169,837,775 |
| | | containing 4 | | forward strand. |
| NR0B2 | ENSG00000131910 | nuclear receptor | SHP | Chromosome 1: |
| | | subfamily 0, group B, | | 26,911,489-26,913,966 |
| | | member 2 | | reverse strand. |
| GRP | ENSG00000134443 | gastrin-releasing | | Chromosome 18: |
| | | peptide | | 59,220,168-59,230,774 |
| | | | | forward strand. |
| CEACAM5 | ENSG00000105388 | carcinoembryonic | CD66e, CEA | Chromosome 19: |
| | | antigen-related cell | | 41,576,273-41,729,798 |
| | | adhesion molecule 5 | | forward strand. |
| SOX21 | ENSG00000125285 | SRY (sex | SOX25 | Chromosome 13: |
| | | determining region | | 94,709,622-94,712,399 |
| | | Y)-box 21 | | reverse strand. |
| OR51E2 | ENSG00000167332 | olfactory receptor, | PSGR | Chromosome 11: |
| | | family 51, subfamily | | 4,680,171-4,697,854 |
| | | E, member 2 | | reverse strand. |
| SEC11C | ENSG00000166562 | SEC11 homolog C, | SEC11L3, | Chromosome 18: |
| | | signal peptidase | SPC21, SPCS4C | 59,139,477-59,158,836 |
| | | complex subunit | | forward strand. |
| BAALC | ENSG00000164929 | brain and acute | | Chromosome 8: |
| | | leukemia, | | 103,140,710-103,230,305 |
| | | cytoplasmic | | forward strand. |
| CCDC40 | ENSG00000141519 | coiled-coil domain | CILD15, FAP172, | Chromosome 17: |
| | | containing 40 | FLJ20753, | 80,036,632-80,100,613 |
| | | | FLJ32021, | forward strand. |
| | | | KIAA1640 | |
| RAB3B | ENSG00000169213 | RAB3B, member | | Chromosome 1: |
| | | RAS oncogene | | 51,907,956-51,990,764 |
| | | family | | reverse strand. |
| RGS17 | ENSG00000091844 | regulator of G-protein | RGS-17, RGSZ2 | Chromosome 6: |
| | | signaling 17 | | 153,004,459-153,131,249 |
| | | | | reverse strand. |
| ABCE1 | ENSG00000164163 | ATP-binding | OABP, RLI, | Chromosome 4: |
| | | cassette, sub-family E | RNASEL1, | 145,097,932-145,129,179 |
| | | (OABP), member 1 | RNASELI, | forward strand. |
| | | | RNS4I | |
| ETS2 | ENSG00000157557 | v-ets avian | | Chromosome 21: |
| | | erythroblastosis virus | | 38,805,307-38,824,955 |
| | | E26 oncogene | | forward strand. |
| | | homolog 2 | | |
| CCDC154 | ENSG00000197599 | coiled-coil domain | C16orf29, | Chromosome 16: |
| | | containing 154 | LOC645811 | 1,434,383-1,444,556 |
| | | | | reverse strand. |
| SPAG6 | ENSG00000077327 | sperm associated | CT141, pf16, | Chromosome 10: |
| | | antigen 6 | Repro-SA-1 | 22,345,445-22,454,224 |
| | | | | forward strand. |
| PON1 | ENSG00000005421 | paraoxonase 1 | ESA, PON | Chromosome 7: |
| | | | | 95,297,676-95,324,707 |
| | | | | reverse strand. |
| TMEM176A | ENSG00000002933 | transmembrane | HCA112, MS4B1 | Chromosome 7: |
| | | protein 176A | | 150,800,403-150,805,120 |
| | | | | forward strand. |
| C1orf127 | ENSG00000175262 | chromosome 1 open | FLJ37118 | Chromosome 1: |
| | | reading frame 127 | | 10,946,471-10,982,037 |
| | | | | reverse strand. |
| IGF2BP2 | ENSG00000073792 | insulin-like growth | IMP-2 | Chromosome 3: |
| | | factor 2 mRNA | | 185,643,739-185,825,056 |
| | | binding protein 2 | | reverse strand. |
| IGFBP5 | ENSG00000115461 | insulin-like growth | | Chromosome 2: |
| | | factor binding protein | | 216,672,105-216,695,525 |
| | | 5 | | reverse strand. |
| FAM84A | ENSG00000162981 | family with sequence | FLJ35392, NSE1 | Chromosome 2: |
| | | similarity 84, | | 14,632,686-14,650,814 |
| | | member A | | forward strand. |
| FOXA2 | ENSG00000125798 | forkhead box A2 | HNF3B | Chromosome 20: |
| | | | | 22,581,005-22,585,455 |
| | | | | reverse strand. |
| HOXA10 | ENSG00000253293 | homeobox A10 | HOX1, HOX1H | Chromosome 7: |
| | | | | 27,170,591-27,180,261 |
| | | | | reverse strand. |
| MYC | ENSG00000136997 | v-myc avian | bHLHe39, c-Myc, | Chromosome 8: |
| | | myelocytomatosis | MYCC | 127,735,434-127,741,434 |
| | | viral oncogene | | forward strand. |
| | | homolog | | |
| NCAM1 | ENSG00000149294 | neural cell adhesion | CD56, NCAM | Chromosome 11: |
| | | molecule 1 | | 112,961,247-113,278,436 |
| | | | | forward strand. |
| NCAM2 | ENSG00000154654 | neural cell adhesion | MGC51008, | Chromosome 21: |
| | | molecule 2 | NCAM21 | 20,998,315-21,543,329 |
| | | | | forward strand. |
| NEUROD1 | ENSG00000162992 | neuronal | BETA2, BHF-1, | Chromosome 2: |
| | | differentiation 1 | bHLHa3, | 181,673,088-181,680,876 |
| | | | MODY6, | reverse strand. |
| | | | NEUROD | |
| KRT8 | ENSG00000170421 | keratin 8, type II | CARD2, CK8, | Chromosome 12: |
| | | | CYK8, K2C8, | 52,897,187-52,949,954 |
| | | | K8, KO | reverse strand. |
| ENO2 | ENSG00000111674 | enolase 2 (gamma, | | Chromosome 12: |
| | | neuronal) | | 6,913,745-6,923,698 |
| | | | | forward strand. |
| AVP | ENSG00000101200 | arginine vasopressin | ADH, ARVP | Chromosome 20: |
| | | | | 3,082,556-3,084,724 |
| | | | | reverse strand. |
| OXT | ENSG00000101405 | oxytocin/neurophysin | OT, OT-NPI, | Chromosome 20: |
| | | I prepropeptide | OXT-NPI | 3,071,620-3,072,517 |
| | | | | forward strand. |
| SYP | ENSG00000102003 | synaptophysin | MRX96 | Chromosome X: |
| | | | | 49,187,804-49,200,259 |
| | | | | reverse strand. |
| CHGA | ENSG00000100604 | chromogranin A | | Chromosome 14: |
| | | | | 92,923,080-92,935,293 |
| | | | | forward strand. |
| CHGB | ENSG00000089199 | chromogranin B | SCG1, SgI | Chromosome 20: |
| | | | | 5,911,430-5,925,361 |
| | | | | forward strand. |
| BCL2 | ENSG00000171791 | B-cell | Bcl-2, PPP1R50 | Chromosome 18: |
| | | CLL/lymphoma 2 | | 63,123,346-63,320,128 |
| | | | | reverse strand. |

The present invention identifies a gene panel (also referred to as “multi-gene panel”, “gene expression panel” or “panel of genes”) whose mRNA expression signature based on in vitro data may serve to identify patients most likely to respond to LSD1 inhibitor containing therapy regimens. The genes listed are characteristic of the SCLC classic phenotype (generally of neuroendocrine origin) to the exclusion of those cell lines of “variant” phenotype. The expression of these genes may have predictive benefit in identifying responder patients of other histological subtypes in additional tumor settings.

It has been found that the mRNA signature is characterized by high expression in responder genes. Responder genes are selected from the group of genes comprising ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB and BCL2.

In a particular embodiment of the invention, responder genese are selected from the group of genes comprising ASCL1, DDC, CEACAM6, LRRIQ4, GRP, NROB2, CEACAM5, SOX21, OR51E2, SEC11C, BAALC, CCDC40, RAB3B, RGS17, ABCE1, ETS2, CCDC154, SPAG6, PON1, TMEM176A, C1orf127, IGFBP5, IGF2BP2, FAM84A, FOXA2, HOXA10.

In a further particular embodiment of the invention, responder genes are selected from the group of genes comprising ASCL1, HOXA10, NCAM1, NCAM2, NEUROD1, DDC, GRP, KRT8, ENO2, AVP, OXT, SYP, CHGA, CHGB, SOX21 and BCL2.

It has further been found, that non-responder lines may be characterized by high levels of the oncogene MYC.

The baseline expression levels of responder genes and non-responder genes listed herein may yield, alone or in combination with one another, a composite score that discriminates between cell lines and patient-derived clinical specimens that are resistant to therapy, and identifies those that are sensitive (responsive) to therapy using an LSD1 inhibitor.

Thus higher levels of responder genes and/or lower expression levels of non-responder genes are indicative for the response to a therapy using an LSD1 inhibitor. Combining the expression levels of several responder and/or non-responder genes may provide a multi-gene signature with improved confidence regarding responsiveness as compared to the readout from single gene expression levels.

The present invention identifies mRNAs associated with and for identifying responses to LSD1 inhibition.

The present invention also relates to a method for identifying sensitivity to LSD1 inhibitor-based therapy.

The present invention also relates to the use of a gene panel in order to determine a patient's response to a neoplastic disease when a patient is to be treated with an LSD1 inhibitor-based therapy.

The present invention also identifies mRNAs expression for monitoring the treatment of neoplastic diseases in a patient with an LSD1 inhibitor.

The present invention also provides the predictive mRNA values in determining the effectiveness of LSD1 inhibitor-based therapy to neoplastic diseases.

One embodiment of the invention provides an in vitro method of identifying a patient having a neoplastic disease as likely to respond to a therapy comprising an LSD1 inhibitor, the method comprising
a) measuring in a sample from the patient the levels of a gene panel, wherein the gene panel comprises one or more genes selected from responder genes and non-responder genes,
b) comparing the levels of the gene panel measured in a) to a reference level, and
c) identifying the patient as more likely to respond to the therapy comprising an LSD1 inhibitor when the levels of the responder genes of the gene panel measured in a) in the sample from the patient are above the reference level, and/or when the levels of the non-responder genes of the gene panel measured in a) in the sample from the patient are below the reference level.

One embodiment of the invention provides an in vitro method of identifying a patient having a neoplastic disease as likely to respond to a therapy comprising an LSD1 inhibitor, the method comprising
a) measuring in a sample from the patient the levels of a gene panel, wherein the gene panel comprises one or more genes selected from responder genes and non-responder genes,
b) calculating a signature score from the measured levels of the gene panel,
c) comparing the signature score calculated to a threshold level, and
d) identifying the patient as more likely to respond to the therapy comprising an LSD1 inhibitor when the signature score is above the threshold level.

Another embodiment of the invention provides an in vitro method of identifying a patient having a neoplastic disease as likely to respond to a therapy comprising an LSD1 inhibitor, the method comprising
a) measuring in a sample from the patient the levels of a gene panel, wherein the gene panel comprises one or more genes selected from responder genes and non-responder genes,
b) comparing the levels of the gene panel measured in a) to a reference level,
c) identifying the patient as more likely to respond to the therapy comprising an LSD1 inhibitor when the levels of the responder genes of the gene panel measured in a) in the sample from the patient are above the reference level, and/or when the levels of the non-responder genes of the gene panel measured in a) in the sample from the patient are below the reference level, and
d) administering an effective amount of LSD1 inhibitor.

One embodiment of the invention provides an in vitro method of identifying a patient having a neoplastic disease as likely to respond to a therapy comprising an LSD1 inhibitor, the method comprising
a) measuring in a sample from the patient the levels of a gene panel, wherein the gene panel comprises one or more genes selected from responder genes and non-responder genes,
b) calculating a signature score from the measured levels of the gene panel,
c) comparing the signature score calculated to a threshold level,
d) identifying the patient as more likely to respond to the therapy comprising an LSD1 inhibitor when the signature score is above the threshold level, and
e) administering an effective amount of LSD1 inhibitor.

Another embodiment of the invention provides an in vitro method of monitoring efficacy of therapy comprising an LSD1 inhibitor in patient having a neoplastic disease, the method comprising
a) measuring in a sample from the patient prior to start of the therapy the levels of a gene panel, wherein the gene panel comprises one or more genes selected from responder genes and non-responder genes,
b) using the levels of the gene panel measured in a) to calculate the patient's signature score prior to start of the therapy,
c) measuring in a sample from the patient after start of the therapy the levels of the gene panel,
d) using the levels of the gene panel measured in c) to calculate the patient's signature score after start of the therapy,
e) comparing the patient's signature score obtained in d) after start of the therapy with the signature score obtained in b) prior to start of the therapy, and
f) identifying the patient as responding to the therapy when the signature score obtained in d) after start of the therapy are higher than the signature score obtained in b) prior to start of the therapy.

Another embodiment of the invention provides an method of treating a patient having a neoplastic disease, the method comprising
a) measuring in a sample from the patient the levels of a gene panel, wherein the gene panel comprises one or more genes selected from responder genes and non-responder genes,
b) comparing the levels of the gene panel measured in a) to a reference level,
c) identifying the patient as more likely to respond to the therapy comprising an LSD1 inhibitor when the levels of the responder genes of the gene panel measured in a) in the sample from the patient are above the reference level, and/or when the levels of the non-responder genes of the gene panel measured in a) in the sample from the patient are below the reference level, and
d) administering an effective amount of LSD1 inhibitor to the patient if likely to respond thereby treating the neoplastic disease.

Another embodiment of the invention provides a method of treating a patient having a neoplastic disease, the method comprising
a) measuring in a sample from the patient the levels of a gene panel, wherein the gene panel comprises one or more genes selected from responder genes and non-responder genes,
b) calculating a signature score from the measured levels of the gene panel,
c) comparing the signature score calculated to a threshold level,
d) identifying the patient as more likely to respond to the therapy comprising an LSD1 inhibitor when the signature score is above the threshold level, and
e) administering an effective amount of LSD1 inhibitor to the patient if likely to respond thereby treating the neoplastic disease.

Another embodiment of the invention provides an LSD1 inhibitor for use in treating a patient having a neoplastic disease, wherein the patient is treated if the levels of the responder genes of a gene panel measured in a sample from the patient are above the reference level, and/or when the levels of the non-responder genes of a gene panel measured in a sample from the patient are below the reference level thereby treating the neoplastic disease.

Another embodiment of the invention provides an in vitro use of gene panel comprising one or more genes selected from responder genes and non-responder genes for assessing a therapy comprising an LSD1 inhibitor in a patient having a neoplastic disease, wherein levels of the responder genes above the reference level, and/or levels of the non-responder genes below the reference level indicate that the patient should be treated with an effective amount of an LSD1 inhibitor.

Another embodiment of the invention provides an in vitro use of gene panel comprising one or more genes selected from responder genes and non-responder genes for identifying a patient having a neoplastic disease as likely to respond to a therapy comprising an LSD1 inhibitor, wherein levels of the responder genes above the reference level, and/or levels of the non-responder genes below the reference level indicate that the patient is more likely to respond to the therapy.

Another embodiment of the invention provides a use of a gene panel comprising one or more genes selected from responder genes and non-responder genes for the manufacture of a diagnostic for assessing a neoplastic disease.

Another embodiment of the invention provides a use of a gene panel comprising one or more genes selected from responder genes and non-responder genes for the manufacture of a diagnostic for assessing a therapy comprising an LSD1 inhibitor in a patient having a neoplastic disease.

Another embodiment of the invention provides a use of a gene panel comprising one or more genes selected from responder genes and non-responder genes for the manufacture of a diagnostic for assessing the likelihood of response of a patient having a neoplastic disease to a therapy comprising an LSD1 inhibitor.

Another embodiment of the invention provides a kit for predicting the likelihood of response to a therapy comprising an LSD1 inhibitor, wherein the kit comprises
a) one or more reagents for measuring the levels of a gene panel in a sample, wherein the gene panel comprises one or more genes selected from responder genes and non-responder genes prior to start of the therapy,
b) one or more comparator molecules to which the levels of a gene panel in the sample are compared.

The term “comparator molecule” refers to a reference standard for normalization across multiple samples. In one embodiment, the comparator molecule is a housekeeping gene used as a standard control for normalization, such as for example actin, TMEM55, or c-abl.

In this application, the term “readout levels” denotes a value which can be in any form of mRNA expression measurement, such as for example expression levels derived from RNA-sequencing such as normalized read counts and RPKM (Reads per Kilobase of Million mapped reads); RT-qPCR; or microarrays.

In this application, the term “normalized read count” denotes the read count which is obtained directly from a RNA-sequencing experiment and which is normalized to make it comparable across experiments.

In this application, the term “normalized expression level” denotes a value which is obtained in a particular kind of expression measurement and which is normalized to make it comparable across experiments (e.g. normalized expression from microarrays, normalized expression from RNA-sequencing).

In one aspect of the invention, the normalized expression level is the normalized read count.

In one aspect of the invention, the levels measured are mRNA expression levels.

In one aspect of the invention, the levels measured are mRNA expression levels derived from RNA-sequencing, RT-qPCR or microarrays.

In one aspect of the invention, the reference level is a standard value from a patient with the same neoplastic disease.

In another embodiment, the reference level is median mRNA expression measured in a population of patients with the same neoplastic disease.

In one aspect of the invention, the reference level for certain genes of the gene panel are as follows (indicated as normalized read counts): ASCL1 (4515.83); DDC (2005.02); GRP (426.01); HOXA10 (10.04).

The reference levels reported above were obtained by selecting the lower normalized read count for the corresponding gene among two small cell lung cancer cell lines C_{S} and C_{R}, wherein C_{S} is the sensitive cell line with the lowest expression of the selected gene, and C_{R} is the resistant cell line with the highest expression of the selected gene.

A signature score as used herein is a gene-based algorithm-derived score (a multi-gene signature) composed of values indicative for up-regulations of responder genes and for down-regulation or copy number variation of non-responder genes.

A signature score larger than a threshold level predicts response to therapy comprising an LSD1 inhibitor. The higher the threshold level for predicting response is selected for the signature score, the higher the specificity obtained. The lower the threshold level for predicting response is selected for the signature score, the higher the sensitivity obtained.

In one embodiment of the invention, the threshold level corresponds to a Signature Score 1 of 0.4 to 0.6, particularly 0.5±20%, most particularly 0.5, wherein the signature score is obtained by partial least square (PLS) analysis using the second principal component:

$$Signature Score 1 = 0.0900693 + ( Normalized expression level of ASCL 1 ) \times 0.00000211296 + ( Normalized expression level of DDC ) \times 0.000000536658 + ( Normalized expression level of GRP ) \times 0.00000297345 + ( Normalized expression level of HOXA 10 ) \times 0.000234721 - ( Copy number variation of MYC ) \times 0.0537056 .$$

In one embodiment of the invention, the threshold level corresponds to a Signature Score 2 of 0.4 to 0.6, particularly 0.5±20%, most particularly 0.5, wherein the signature score is obtained by partial least square (PLS) analysis using the first principal component:

$$Signature Score 2 = 0.483918 + ( Normalized expression level of ASCL 1 ) \times 0.00000188066 + ( Normalized expression level of DDC ) \times 0.00000188066 + ( Normalized expression level of GRP ) \times 0.00000352033 - ( Copy number variation of MYC ) \times 0.0407898 .$$

In one embodiment of the invention, the threshold level corresponds to a Signature Score 3 of 0.4 to 0.6, particularly 0.5±20%, most particularly 0.5, wherein the signature score is obtained by partial least square (PLS) analysis using the first principal component:

$$Signature Score 3 = 0.393569 + ( Normalized expression level of ASCL 1 ) \times 0.00000182731 + ( Normalized expression level of DDC ) \times 0.00000189664 + ( Normalized expression level of GRP ) \times 0.00000342046 .$$

A signature score above the threshold level indicates a high likelihood of response to treatment with an LSD1 inhibitor, whereas a signature score below said level indicates a lower likelihood to respond to such treatment. A higher score is associated with higher mRNA expression of ASCL1, DDC, GRP and HOXA10, and with lower copy number variations in MYC.

In one embodiment of the invention, the reference level is the threshold level of a signature score.

In one embodiment of the invention, the signature score to predict response to therapy comprising an LSD1 inhibitor may be obtained by performing the following steps:
a. Select a gene panel which comprises m genes, wherein m is an integer greater than 1, selected among the genes disclosed in Table 6, and optionally HOXA10 and MYC.
b. Select a set of one or more sensitive and a set of one or more resistant cancer cell lines, particularly originating from neuroendocrine tumors such as small cell lung cancer (SCLC), as for example described in Table 3. Alternatively select a set of one or more classic and set of one or more variant small cell lung cancer cell lines.
c. Generate an n×m matrix, wherein m is as defined above and n is the total number of small cell lung cancer cell lines selected. The matrix contains expression levels of the selected genes (and/or copy number variations in case of the MYC). Gene expression levels may be reported as RPKM or as normalized read counts.
d. Generate a response vector of size n, which describes each cell line as being sensitive (“S”) or resistant (“R”), as defined in Table “3”. Alternatively, this vector may describe each cell line as being of “classic” (C”) or “variant” (V”) subtype.
e. Apply a machine learning algorithm for classification of the matrix described above in point c. Examples of such machine learning algorithms include, but are not limited to, decision trees, support-vector machines, neural networks, nearest neighbor analysis, naïve Bayes, random forest, partial least square, etc.
f. Perform appropriate cross-validation using either cell lines included in the analysis and/or cell lines not included in the analysis to optimize the model's predictive power.
g. Select a function f(x), as appropriate for the machine learning algorithm selected, to obtain a signature score y (y=f(x)). This function f(x) comprises a set of coefficients a1 . . . ap calculated by the machine learning algorithm (where p is the number of coefficients selected by a given algorithm) and gene expression levels (x1 . . . xm) of the genes selected.
h. Select a threshold, as proposed by the machine learning method, to determine whether the signature score predicts sensitivity or resistance to an LSD1 inhibition therapy.

In one embodiment of the invention the gene panel comprises one or more genes selected from the group of MYC, ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB and BCL2.

In a particular embodiment of the invention the gene panel comprises one or more genes selected from the group of MYC, ASCL1, DDC, CEACAM6, LRRIQ4, GRP, NROB2, CEACAM5, SOX21, OR51E2, SEC11C, BAALC, CCDC40, RAB3B, RGS17, ABCE1, ETS2, CCDC154, SPAG6, PON1, TMEM176A, C1orf127, IGFBP5, IGF2BP2, FAM84A, FOXA2, HOXA10.

In a particular embodiment of the invention the gene panel comprises one or more genes selected from the group of ASCL1, MYC, HOXA10, DDC, GRP, NCAM1, NCAM2, NEUROD1, KRT8, ENO2, AVP, OXT, SYP, CHGA, CHGB, SOX21 and BCL2.

In a particular embodiment of the invention the gene panel comprises one or more genes selected from the group of ASCL1, MYC, HOXA10, DDC, GRP, NCAM1, NCAM2, NEUROD1, SOX21 and BCL2.

In a particular embodiment of the invention the gene panel comprises two, three, four or five genes selected from the group of ASCL1, MYC, HOXA10, DDC, GRP, NCAM1, NCAM2, NEUROD1, SOX21 and BCL2.

In a particular embodiment of the invention the gene panel comprises one or more genes selected from the group of ASCL1, MYC, HOXA10, DDC and GRP.

In a particular embodiment of the invention the gene panel comprises two, three, four or five genes selected from the group of ASCL1, MYC, HOXA10, DDC and GRP.

In a particular embodiment of the invention the gene panel comprises one or more genes selected from the group of ASCL1, MYC and HOXA10.

In a particular embodiment of the invention the gene panel comprises the ASCL1 gene.

In a particular embodiment of the invention the gene panel comprises the MYC gene.

In a particular embodiment of the invention the gene panel comprises the HOXA10 gene.

In a particular embodiment of the invention the gene panel comprises the DDC gene.

In a particular embodiment of the invention the gene panel comprises the GRP gene.

In a particular embodiment of the invention the gene panel consists of one, two, three, four or five genes.

In a particular embodiment of the invention the gene panel consists of two, three or four genes.

In one embodiment of the invention the responder genes are selected from the group of ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB and BCL2.

In a particular embodiment of the invention, responder genese are selected from the group of ASCL1, DDC, CEACAM6, LRRIQ4, GRP, NROB2, CEACAM5, SOX21, OR51E2, SEC11C, BAALC, CCDC40, RAB3B, RGS17, ABCE1, ETS2, CCDC154, SPAG6, PON1, TMEM176A, C1orf127, IGFBP5, IGF2BP2, FAM84A, FOXA2, HOXA10.

In a particular embodiment of the invention the responder genes are selected from the group of ASCL1, HOXA10, DDC, GRP, NCAM1, NCAM2, NEUROD1, KTR8, ENO2, AVP, OXT, SYP, CHGA, CHGB, SOX21 and BCL2.

In a particular embodiment of the invention the non-responder genes are selected from MYC.

In one aspect of the present invention, the LSD1 inhibitor is selected from a compound as described in WO 2011/131697 (A1), WO 2012135113 (A2) and WO 2013/057322 (A1).

In a particular embodiment of the invention the LSD1 inhibitor is selected from the list of:
4-[[4-[[[(1R,2S)-2-phenylcyclopropyl]amino]methyl]-1-piperidinyl]methyl]-benzoic acid (trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-diamine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine,
and a pharmaceutically acceptable salt thereof.

In a particular embodiment of the invention the LSD1 inhibitor is GSK2879552 [CAS Reg. No. 1401966-69-5], also known as 4-[[4-[[[(1R,2S)-2-phenylcyclopropyl]amino]methyl]-1-piperidinyl]methyl]-benzoic acid, or a pharmaceutically acceptable salt thereof.

In a particular embodiment of the invention the LSD1 inhibitor is selected from the list of:
(trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-diamine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine,
and a pharmaceutically acceptable salt thereof.

In a particular embodiment of the invention the LSD1 inhibitor is (trans)-N1-((1R,2S)-2-phenylcyclopropyl)cyclohexane-1,4-diamine [CAS Reg. No. 1431304-21-0] or a pharmaceutically acceptable salt thereof.

In a particular embodiment of the invention the LSD1 inhibitor is (trans)-N1-((1R,2S)-2-phenylcyclopropyl)cyclohexane-1,4-diamine [CAS Reg. No. 1431304-21-0] or a hydrochloride salt thereof.

In a particular embodiment of the invention the LSD1 inhibitor is (trans)-N1-((1R,2S)-2-phenylcyclopropyl)cyclohexane-1,4-diamine bis-hydrochloride [CAS Reg. No. 1431303-72-8].

In a particular embodiment of the invention the LSD1 inhibitor is administered to a patient in need thereof orally, such as an oral solution.

Measurements may be taken from a blood specimen, a bone marrow specimen or a fresh frozen or formalin-fixed paraffin embedded primary human tumor specimen.

As described above, LSD1 inhibitors have been described for use in the treatment of patients having a neoplastic disease.

In a particular embodiment of the invention the neoplastic disease that is potentially treatable based on the desired LSD1 clinical response is a cancer, particularly a cancer selected from the group consisting of breast cancer, prostate cancer, cervical cancer, ovarian cancer, gastric cancer, colorectal cancer (i.e. including colon cancer and rectal cancer), pancreatic cancer, liver cancer, brain cancer, neuroendocrine cancer, lung cancer, kidney cancer, hematological malignancies, melanoma and sarcomas.

In a particular embodiment of the invention the cancer that is potentially treatable based on the LSD1 response is selected from the group consisting of hematological malignancies, neuroendocrine cancer, breast cancer, cervical cancer, ovarian cancer, colorectal cancer, melanoma and lung cancer.

In a particular embodiment of the invention the neoplastic disease is a cancer selected from the group consisting of blood cancer or lung cancer, more particularly acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

In a particular embodiment of the invention the neoplastic disease is a blood cancer or lung cancer selected from the group of acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

In a particular embodiment of the invention the neoplastic disease is a cancer is selected from the group consisting of acute myeloid leukemia (AML), non-Hodgkin's lymphoma, small cell lung cancer (SCLC), thyroid cancer, and melanoma.

In a particular embodiment of the invention the neoplastic disease is a cancer selected from the group consisting of acute myeloid leukemia (AML), thyroid cancer, melanoma, or small cell lung cancer (SCLC).

In a particular embodiment of the invention the neoplastic disease is a cancer selected from the group consisting of acute myeloid leukemia (AML) and small cell lung cancer (SCLC).

In a particular embodiment of the invention the neoplastic disease is neuroendocrine cancer.

In a particular embodiment of the invention the neoplastic disease is lung cancer.

In a particular embodiment of the invention the neoplastic disease is small cell lung cancer (SCLC).

### DESCRIPTION OF THE DRAWINGS

FIG. 1: Principal component analysis score plot for principal component 1 (t[1], x-axis) and principal component 2 (t[2], y-axis) separates classic cell lines (C, black) from variant cell lines (V, gray) according to Example 1.

FIG. 2: Heat Map showing mRNA expression (as z-scores) for the gene panel of Example 2 comprising the genes of Table 5, Table 6 and MYC. These genes best predict response to an LSD1 inhibition therapy in the 19 cell lines of Table 3. Higher z-scores correlate with better sensitivity.

FIG. 3: Heat Map showing mRNA expression (as z-scores) for the neuroendocrine genes of Example 3 in the 19 cell lines of Table 3. Sensitive cell-lines display a stronger expression (higher z-score) of such neuroendocrine markers.

FIG. 4: Signature scores obtained by PLS analysis using the second principal component according to Example 4. Cell lines with score_1>0.5 are more likely to be sensitive to an LSD1 inhibition therapy.

FIG. 5: Signature scores obtained by PLS analysis using the first principal component according to Example 4. Cell lines with score_2>0.5 are more likely to be sensitive to an LSD1 inhibition therapy.

FIG. 6: Signature scores obtained by PLS analysis using the first principal component according to Example 4. Cell lines with score_3>0.45 are more likely to be sensitive to an LSD1 inhibition therapy.

FIG. 7: in vivo tumor growth inhibition of (trans)-N1-((1R,2S)-2-phenylcyclopropyl)cyclohexane-1,4-diamine in classic (C) cell line H-510A sensitive (S) to therapy comprising an LSD1 inhibitor.

### EXAMPLES

The following examples 1 to 4 are provided for illustration of the invention. They should not be considered as limiting the scope of the invention, but merely as being representative thereof.

### Methods

Expression Data

Expression data were obtained from whole transcriptomic RNA sequencing (RNA-seq) by Illumina, Inc. (San Diego, Calif.). The Illumina HiSeq machine generates raw base calls in reads of 50 or 100 bp length, which are subjected to several data analysis steps. The RNA-seq is conducted at 40 to 50 million reads per sample. This number provides relatively high sensitivity to detect low-expressed genes while allowing for cost-effective multiplexing of samples. RNA is prepared by standard kits and RNA libraries by polyA TruSeq Illumina kits. 100 ng of mRNA per cell line is used for each RNA-seq reaction. A number of quality control procedures are applied to the RNA-seq data for each sample. The Illumina HiSeq software reports the total number of clusters (DNA fragments) loaded in each lane, percent passing sequencing quality filters (which identifies errors due to overloading and sequencing chemistry), a phred quality score for each base of each sequence read, overall average phred scores for each sequencing cycle, and overall percent error (based on alignment to the reference genome). For each RNA-seq sample, the percentage of reads that contain mitochondrial and ribosomal RNA is calculated. The FASTQC package is used to provide additional QC metrics (base distribution, sequence duplication, overre resented sequences, and enriched kmers) and a graphical summary. Raw reads were aligned against the human genome (hg19) using GSNAP and recommended options for RNASeq data. In addition to the genome sequence, GSNAP is given a database of human splice junctions and transcripts based on Ensembl v73. Resulting SAM files are then converted to sorted BAM files using Samtools. Gene expression values are calculated both as RPKM values following (Mortazavi et al. Nat Methods (2008) 5(7):621-8) and as read counts. Normalized read counts were obtained using the R package DESeq2.

Copy Number Variations (CNV)

To obtain copy number variation data genomic DNA were extracted and array CGH analysis were performed by Roche NimbleGen (Madison, Wis.) using their standard protocols. Normalized signal intensities and copy number changes were obtained using the segMNT algorithm. CGH microarrays contain isothermal, 45- to 85-mer oligonucleotide probes that are synthesized directly on a silica surface using light-directed photochemistry (Selzer et al., Genes Chromosomes Cancer (2005) 44(3):305-319). The genomic DNA samples are randomly fragmented into lower molecular weight species and differentially labeled with fluorescent dyes.

Principal Component Analysis

Principal component analysis was carried out with Simca v 14 (Umetrics AB, Umeå Sweden).

Differential Gene Expression Analysis

Differential gene expression analysis used to generate data in Table 6 was carried out with the R package DESeq2 starting from raw read counts for 19 cell lines.

Heat Maps

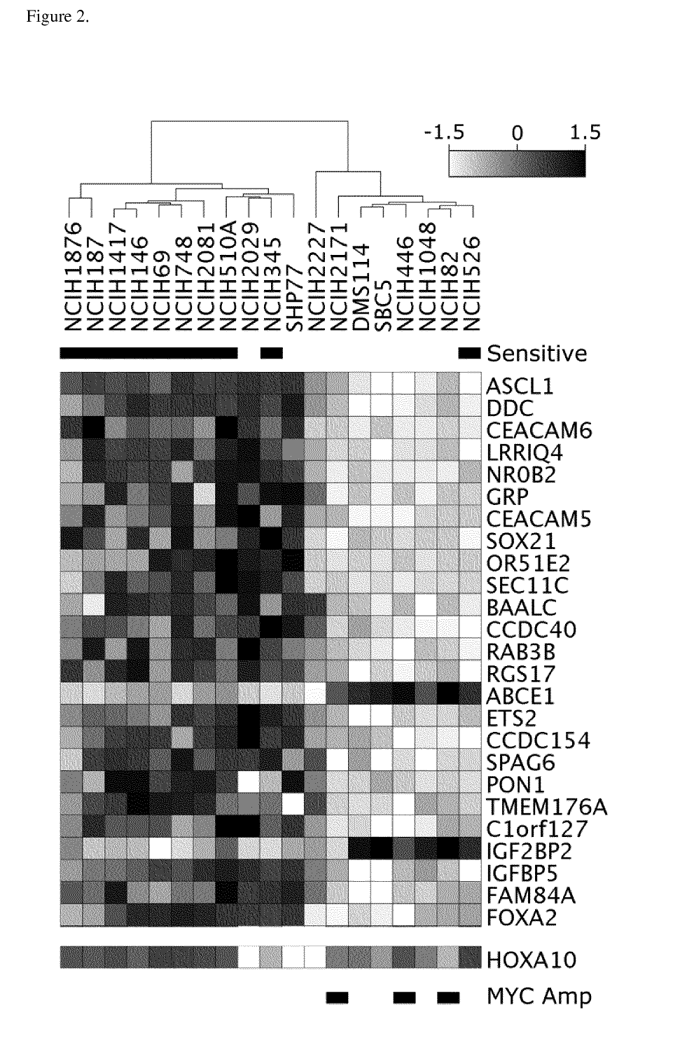

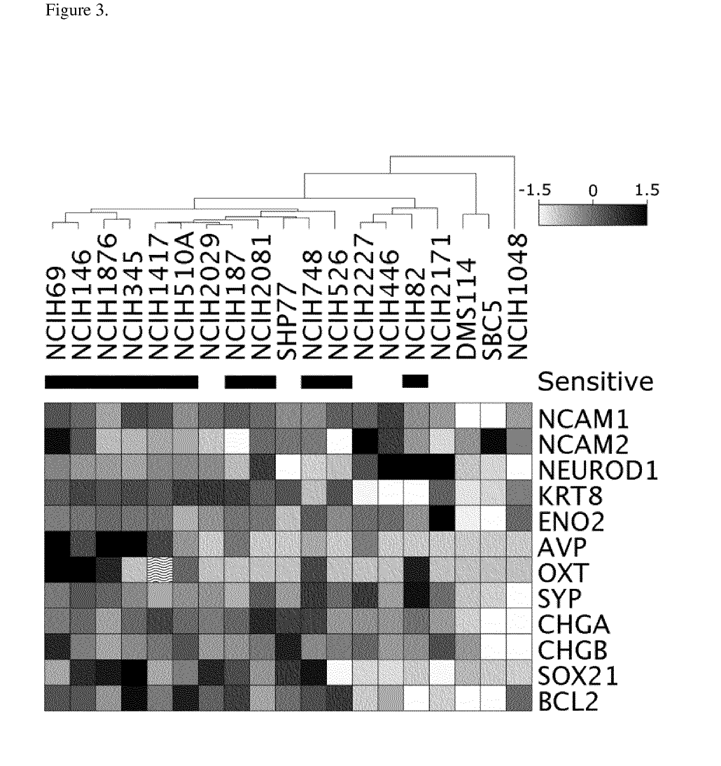

Heat maps (as in FIGS. 2 and 3) were generated using GenePattern v 3.9.4 (Reich M. et al., Nature Genetics (2006) 38(5): 500-501) to visualize color-coded gene expression levels. GenePattern takes in input the logarithm of normalized read counts (as reported in Table 8) plus one and applies a row-based normalization which consists of calculating z-scores for all expression levels of a given gene across the cell lines tested. A z-score of 0 corresponds to the mean of a distribution, and positive or negative value represent normalized gene expression levels above or below the mean, respectively. The color mapping capped the z-score range from −1.5 to +1.5, that is, z-scores above +1.5 are displayed in black and z-scores below −1.5 are in white. Intermediate values are displayed in different shades of gray. Gene Pattern performs hierarchical clustering to group and sort cell lines based on their gene expression profile.

### Example 1. Cell Response to LSD1 Inhibition

The compound potency determination was performed by culturing 19 small cell lung cancer cell lines (of various solid and non-solid tumor origins) for 4 days at 37 degrees C. at 5% CO₂ in humidified incubators in the presence of serially diluted (trans)-N1-((1R,2S)-2-phenylcyclopropyl)cyclohexane-1,4-diamine bis-hydrochloride.

As a positive control for cytotoxicity the Hsp90 inhibitor 17-N-allylamino-17-demethoxygeldanamycin (17-AAG, a geldanamycin analogue) was used as positive control in serial dilution. Each of the cell lines was propagated and tested in distinct optimized media as recommended by ATCC or cell line source.

Small cell lung cancer cell lines can be categorized as “classic” or “variant”, based on their enzymatic activities, cellular morphologies, and growth phenotypes (Desmond et al., Cancer Res (1985) 45(6):2913-2923; Shoemaker R. H., Nature Reviews Cancer (2016) 6:813-823). Classic cells lines express elevated levels of L-dopa decarboxylase, bombesin-like immunoreactivity, neuron-specific enolase, and the brain isozyme of creatine kinase; variant cell lines continue to express neuron-specific enolase and the brain isozyme of creatine kinase, but have undetectable levels of L-dopa decarboxylase and bombesin-like immunoreactivity. Unlike classic cell lines, some variant cell lines are amplified for and have increased expression of the c-myc (MYC)oncogene.

Some cell lines exhibit features specific to both a classic and variant subtype. For example, SHP-77 has biochemical properties of classic SCLC (e.g. elevated levels of L-dopa decarboxylase and bombesin-like immunoreactivity) but the morphology of a variant. According to the literature, SHP-77 is considered classic based on its biochemical profile but variant based on its morphology and growth characteristic.

For NCI-H2029 and SBC-5 no subtype is reported in literature however their transcriptomic profile (mRNA expression levels of DDC/GRP) clearly shows their class membership which is provided in brackets in Table 3.

Depending on their responses to (trans)-N1-((1R,2S)-2-phenylcyclopropyl)cyclohexane-1,4-diamine bis-hydrochloride, cell lines are classified as either “sensitive” [5], defined as having EC50<0.05 μM, or “resistant”, defined as having EC50>=0.05 μM [R].

Cell-based response to (trans)-N1-((1R,2S)-2-phenylcyclopropyl)cyclohexane-1,4-diamine bis-hydrochloride was greater in classic SCLC cell lines compared to variant SCLC cell lines (p-value 0.0055 Table 3). Out of the 19 SCLC cell lines tested, 9 out of 11 classic cell lines [C] are sensitive [5], and 7 out of 8 variant cell lines [V] are resistant [R] (Table 4).

The variant and classic subtypes predict response to an LSD1 inhibitor therapy with a sensitivity of 82% and specificity of 88%.

Higher copy number variations (CNV) in the MYC gene (Ensemble Gene ID: ENSG00000136997) are associated with small cell lung cancer of variant subtype (V) (Am J Pathol. 1988 July; 132(1): 13-17). Indeed, among the 19 cell lines here described, high copy number variations of the MYC gene (CNV>>2) were found exclusively in cell lines with a variant subtype (NCI-H2171, NCI-H446, NCI-H82, see Table 3). Furthermore, all three cell lines with high copy number variations of MYC were resistant to LSD1 inhibition, indicating that the presence of MYC amplification can predict resistance (R) to an LSD1 inhibition therapy.

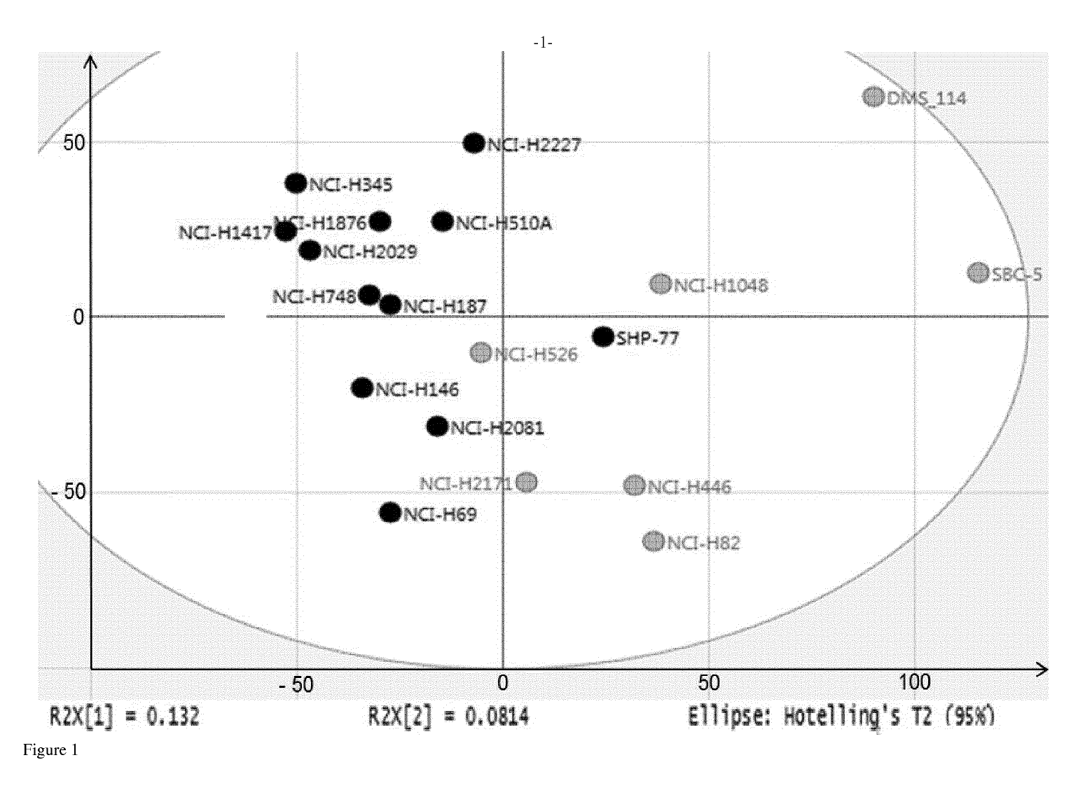

Principal component analysis carried out from RNA-seq data for the cell lines of Table 3 surprisingly revealed that classic and variant SCLC cell lines form distinct clusters. (FIG. 1).

### Example 2. Gene Panel to Predict Response to LSD1 Inhibition

Differential gene expression analysis between two resistant cell lines that have features of a classic subtype (SHP-77 and NCI-2029) and classic and variant cell lines which are sensitive (NCI-H1876, NCI-H69, NCI-H510A, NCI-H146, NCI-H187, NCI-H2081, NCI-H345, NCI-H526, NCI-H748) interestingly revealed that lower mRNA expression levels of HOXA10 correlate with resistance to an LSD1 inhibition therapy (Table 5). This suggests that low levels of HOXA10 mRNA may predict resistance to an LSD1 inhibition therapy even in the presence of a classic phenotype.

A predictive mRNA expression signature of response to an LSD1 inhibition therapy was defined by selecting top differentially expressed genes between classic and variant cell lines (Table 6). Based on adjusted p-values, DDC (adjusted p-value 4.37E-23), which encodes the enzyme L-dopa decarboxylase, and GRP (adjusted p-value 5.19E-14), which encodes bombesin-like immunoreactivity peptides rank as second and sixth most differentially expressed genes. The most differentially expressed gene is ASCL1 (adjusted p-value 2.6E-23). ASCL1 is a transcription factor required for proper development of pulmonary neuroendocrine cells, and is essential for the survival of a majority of lung cancers (Augustyn et al., Proc Natl Acad Sci USA (2014) 111(41):14788-93).

As discussed in Example 1 above, MYC amplification can predict resistance to LSD1 inhibition therapy.

Table 7 lists normalized read counts of DDC, GRP, and ASCL1 across the 19 cell lines of Table 2 described while Table 8 lists the corresponding z-scores.

The heat map of FIG. 2 visually shows that sensitive cell lines can be distinguished from resistant cell lines based on mRNA expression levels of genes listed in Table 6, and based on expression levels of HOXA10 and copy number variations of MYC.

TABLE 5
Principal component analysis for HOXA10 carried out from RNA-seq
data for selected cell lines (*http://www.ensembl.org/, Cunningham
F. et al., Nucl. Acids Res. (2015) 43(D1): D662-D669).

| | | | log2Fold | |
|---|---|---|---|---|
| Ensembl Gene ID* | Gene | baseMean | Change | pvalue |
| ENSG00000253293 | HOXA10 | 2717.58 | 8.21 | 7.45E−023 |

TABLE 6
Genes sorted according to pvalue obtained through principal
component analysis carried out from RNA-seq data for selected
cell lines (*http://www.ensembl.org/, Cunningham F. et
al., Nucl. Acids Res. (2015) 43(D1): D662-D669).

| | | | log2Fold | |
|---|---|---|---|---|
| Ensembl Gene ID* | Gene | baseMean | Change | pvalue |
| ENSG00000139352 | ASCL1 | 43665.33 | 6.82 | 2.62E−023 |
| ENSG00000132437 | DDC | 15817.8 | 6.42 | 4.37E−023 |
| ENSG00000086548 | CEACAM6 | 210.89 | 6.34 | 1.23E−017 |
| ENSG00000188306 | LRRIQ4 | 90.81 | 5.1 | 4.61E−016 |
| ENSG00000131910 | NR0B2 | 600.58 | 6.35 | 5.15E−015 |
| ENSG00000134443 | GRP | 6711.45 | 6.52 | 5.19E−014 |
| ENSG00000105388 | CEACAM5 | 1788.17 | 6.22 | 9.23E−014 |
| ENSG00000125285 | SOX21 | 523.59 | 5.88 | 2.29E−013 |
| ENSG00000167332 | OR51E2 | 3047.56 | 6.39 | 3.37E−013 |
| ENSG00000166562 | SEC11C | 36139.18 | 3.33 | 5.01E−013 |
| ENSG00000164929 | BAALC | 1833.4 | 4.33 | 1.66E−012 |
| ENSG00000141519 | CCDC40 | 2309.83 | 2.26 | 2.07E−012 |
| ENSG00000169213 | RAB3B | 28247.78 | 3.64 | 2.80E−012 |
| ENSG00000091844 | RGS17 | 2783.99 | 3.2 | 3.72E−012 |
| ENSG00000164163 | ABCE1 | 13643.12 | −1.08 | 4.99E−012 |
| ENSG00000157557 | ETS2 | 11829.42 | 3.06 | 5.19E−012 |
| ENSG00000197599 | CCDC154 | 1198.98 | 4.61 | 7.21E−012 |
| ENSG00000077327 | SPAG6 | 767.39 | 5.34 | 7.85E−012 |
| ENSG00000005421 | PON1 | 334.17 | 5.15 | 1.53E−011 |
| ENSG00000002933 | TMEM176A | 3224.04 | 5.38 | 7.65E−011 |
| ENSG00000175262 | C1orf127 | 596.15 | 5.04 | 1.19E−010 |
| ENSG00000073792 | IGF2BP2 | 2414.53 | −5.17 | 1.28E−010 |
| ENSG00000115461 | IGFBP5 | 86866.7 | 4.41 | 1.38E−010 |
| ENSG00000162981 | FAM84A | 4954.8 | 3.93 | 1.45E−010 |
| ENSG00000125798 | FOXA2 | 4530.46 | 5.12 | 1.71E−010 |

TABLE 7
Normalized read counts from mRNA expression levels

| Cell Line | ASCL1 | DDC | GRP | HOXA10 |
|---|---|---|---|---|
| NCI-H1417 | 42666.4 | 16161.1 | 10935.2 | 3327.72 |
| NCI-H1876 | 34116.3 | 986.718 | 43.7461 | 2779.5 |
| NCI-H69 | 19902.1 | 25773.6 | 3256.24 | 4271.2 |
| NCI-H510A | 79879.7 | 19456.3 | 27861 | 2730.12 |
| NCI-H2227 | 4515.83 | 2005.02 | 645.86 | 2.59381 |
| NCI-H2029 | 127171 | 39070.6 | 1800.43 | 10.0396 |
| NCI-H146 | 59238.2 | 45308.8 | 426.015 | 2126.39 |
| NCI-H187 | 71323.6 | 4363.62 | 130.681 | 2448.85 |
| NCI-H2081 | 69670.9 | 29683.5 | 2.97459 | 3423.76 |
| NCI-H345 | 81805.8 | 16935.7 | 30601.3 | 263.11 |
| SHP-77 | 115523 | 71808.9 | 39002.6 | 4.72759 |
| NCI-H748 | 122007 | 27938.7 | 12773.8 | 3940.53 |
| DMS-114 | 59.1696 | 16.3227 | 12.242 | 1462.92 |
| NCI-H1048 | 38.9626 | 90.2292 | 0 | 1168.88 |
| NCI-H2171 | 1115.78 | 368.976 | 0 | 1248.61 |
| NCI-H446 | 13.1805 | 32.0098 | 11.2976 | 2818.75 |
| NCI-H82 | 577.05 | 486.304 | 9.30725 | 221.047 |
| SBC5 | 4.51028 | 13.5308 | 0 | 617.908 |
| NCI-H526 | 11.9576 | 38.2644 | 4.78305 | 4091.9 |

TABLE 8
Z-scores generated by GenePattern
from normalized mRNA read counts

| Cell Line | ASCL1 | DDC | GRP | HOXA10 |
|---|---|---|---|---|
| NCI-H1417 | 0.63 | 0.69 | 1.09 | 0.67 |
| NCI-H1876 | 0.57 | −0.24 | −0.34 | 0.6 |
| NCI-H69 | 0.42 | 0.85 | 0.78 | 0.78 |
| NCI-H510A | 0.8 | 0.76 | 1.34 | 0.59 |
| NCI-H2227 | 0.02 | 0 | 0.35 | −2.31 |
| NCI-H2029 | 0.93 | 0.99 | 0.62 | −1.82 |
| NCI-H146 | 0.72 | 1.04 | 0.25 | 0.48 |
| NCI-H187 | 0.77 | 0.26 | −0.06 | 0.54 |
| NCI-H2081 | 0.77 | 0.9 | −0.98 | 0.69 |
| NCI-H345 | 0.81 | 0.71 | 1.36 | −0.43 |
| SHP-77 | 0.9 | 1.19 | 1.43 | −2.11 |
| NCI-H748 | 0.92 | 0.88 | 1.13 | 0.75 |
| DMS-114 | −1.16 | −1.59 | −0.66 | 0.32 |
| NCI-H1048 | −1.27 | −1.04 | −1.34 | 0.22 |
| NCI-H2171 | −0.36 | −0.57 | −1.34 | 0.25 |
| NCI-H446 | −1.55 | −1.38 | −0.68 | 0.6 |
| NCI-H82 | −0.54 | −0.48 | −0.73 | −0.51 |
| SBC5 | −1.81 | −1.65 | −1.34 | −0.06 |
| NCI-H526 | −1.58 | −1.32 | −0.88 | 0.76 |

### Example 3. Neuroendocrine Gene Panel to Predict Response to LSD1 Inhibition

mRNA expression levels for a second set of genes according to Table 9(NCAM1, NCAM2, NEUROD1, KRT8, ENO2, AVP, OXT, SYP, CHGA, CHGB, SOX21, BCL2) that includes genes representative of a neuroendocrine phenotype and that are used as immunohistochemical markers for diagnosing lung neuroendocrine tumors are strongly downregulated in resistant cell lines DMS114, SBC5, and NCI-H1048, as illustrated in FIG. 3. This is an agreement with our hypothesis that an LSD1 inhibition therapy stops cellular growth in tumors of neuroendocrine origin.

Tables 10A and 10B list normalized read counts of the genes of Table 9 across the 19 cell lines of Table 2 described.

TABLE 9
Genes of the second neuroendocrine gene panel
(*http://www.ensembl.org/, Cunningham F. et al.,
Nucl. Acids Res. (2015) 43(D1): D662-D669).

| Ensembl Gene ID* | Gene |
|---|---|
| ENSG00000149294 | NCAM1 |
| ENSG00000154654 | NCAM2 |
| ENSG00000162992 | NEUROD1 |
| ENSG00000170421 | KRT8 |
| ENSG00000111674 | ENO2 |
| ENSG00000101200 | AVP |
| ENSG00000101405 | OXT |
| ENSG00000102003 | SYP |
| ENSG00000100604 | CHGA |
| ENSG00000089199 | CHGB |
| ENSG00000125285 | SOX21 |
| ENSG00000171791 | BCL2 |

TABLE 10A
Normalized read counts from mRNA expression levels.

| Cell Line | NCAM1 | NCAM2 | NEUROD1 | KRT8 | ENO2 | AVP |
|---|---|---|---|---|---|---|
| NCI-H1417 | 52961.1 | 230.0 | 257.7 | 32261.1 | 32287.3 | 5.8 |
| NCI-H1876 | 12131.4 | 111.0 | 143.4 | 36460.8 | 37021.4 | 33.2 |
| NCI-H69 | 53702.4 | 16861.8 | 295.0 | 28560.6 | 28765.0 | 18.6 |
| NCI-H510A | 21010.6 | 197.4 | 255.2 | 67662.7 | 11901.4 | 1.7 |
| NCI-H2227 | 42956.2 | 32469.4 | 1273.6 | 181.6 | 35558.6 | 2.6 |
| NCI-H2029 | 37343.8 | 70.3 | 244.3 | 76401.1 | 22753.0 | 0.0 |
| NCI-H146 | 39176.8 | 1929.1 | 173.4 | 50190.4 | 32430.6 | 5.5 |
| NCI-H187 | 47022.6 | 8.5 | 31.3 | 61809.4 | 32195.9 | 2.8 |
| NCI-H2081 | 37569.1 | 1279.1 | 2427.3 | 26842.7 | 32137.5 | 0.0 |
| NCI-H345 | 62260.5 | 131.6 | 96.7 | 46256.4 | 32848.5 | 45.6 |
| SHP-77 | 21787.1 | 990.4 | 0.0 | 35148.0 | 8851.6 | 0.0 |
| NCI-H748 | 21844.8 | 892.7 | 12.1 | 1508.8 | 44468.6 | 0.9 |
| DMS-114 | 95.9 | 512.1 | 18.4 | 377.5 | 3260.5 | 0.0 |
| NCI-H1048 | 14740.2 | 760.8 | 0.0 | 12726.4 | 38304.4 | 0.0 |
| NCI-H2171 | 16524.2 | 35.4 | 60402.8 | 26223.8 | 212034.0 | 0.0 |
| NCI-H446 | 79657.4 | 3747.0 | 19164.5 | 45.2 | 36229.5 | 0.0 |
| NCI-H82 | 20878.5 | 437.4 | 34283.3 | 27.9 | 22702.7 | 0.0 |
| SBC-5 | 130.8 | 19026.6 | 9.0 | 640.5 | 160.1 | 0.0 |
| NCI-H526 | 44561.3 | 0.0 | 23.9 | 38233.3 | 24912.5 | 0.0 |

TABLE 10B
Normalized read counts from mRNA expression levels.

| Cell Line | OXT | SYP | CHGA | CHGB | SOX21 | BCL2 |
|---|---|---|---|---|---|---|
| NCI-H1417 | NA | 6220.2 | 44388.5 | 11152.1 | 20.4 | 6170.7 |
| NCI-H1876 | 4.2 | 13216.2 | 7061.0 | 3968.7 | 1201.4 | 4126.7 |
| NCI-H69 | 9.5 | 10950.9 | 16527.4 | 52724.6 | 20.9 | 10853.4 |
| NCI-H510A | 1.8 | 9116.9 | 22660.3 | 20808.2 | 79.1 | 27378.7 |
| NCI-H2227 | 0.0 | 19962.0 | 11537.3 | 14927.4 | 2.6 | 1136.1 |
| NCI-H2029 | 0.0 | 8905.1 | 16397.9 | 5776.1 | 786.4 | 8687.6 |
| NCI-H146 | 16.9 | 14940.0 | 22829.6 | 9597.3 | 660.2 | 10340.5 |
| NCI-H187 | 0.0 | 5696.0 | 23923.2 | 6804.0 | 264.2 | 14934.6 |
| NCI-H2081 | 0.0 | 14334.6 | 79374.1 | 10934.6 | 44.6 | 2778.3 |
| NCI-H345 | 0.0 | 9686.8 | 22971.1 | 7702.7 | 4953.5 | 39332.3 |
| SHP-77 | 0.0 | 7861.2 | 47453.1 | 61511.4 | 480.6 | 7364.0 |
| NCI-H748 | 2.7 | 19958.6 | 46176.5 | 7932.8 | 1408.9 | 11595.8 |
| DMS-114 | 0.0 | 4299.0 | 1897.5 | 6161.8 | 10.2 | 185.7 |
| NCI-H1048 | 0.0 | 260.4 | 16.4 | 6.2 | 4.1 | 8063.2 |
| NCI-H2171 | 0.0 | 12335.6 | 9407.4 | 23159.9 | 0.0 | 1065.6 |
| NCI-H446 | 0.0 | 7403.7 | 10702.6 | 5688.3 | 1.9 | 2398.9 |
| NCI-H82 | 4.7 | 31714.5 | 19382.3 | 7303.9 | 4.7 | 148.9 |
| SBC-5 | 0.0 | 3642.1 | 311.2 | 203.0 | 4.5 | 306.7 |
| NCI-H526 | 0.0 | 12538.8 | 9920.1 | 9877.0 | 0.0 | 16511.1 |

### Example 4. Signature Scores to Predictive Response to LSD1 Inhibition

Normalized expression levels (Norm_read_count) of ASCL1, DDC, GRP, and HOXA10 and MYC copy number variations (Copy_number_variation) were used to generate a gene signature to predict response to an LSD1 inhibition therapy as follows:

A score was generated from the following equation, obtained by partial least square (PLS) analysis using the second principal component:

$$Signature Score 1 = 0.0900693 + Norm\_read \_count ( ASCL 1 ) \times 0.00000211296 + Norm\_read \_count ( DDC ) \times 0.000000536658 + Norm\_read \_count ( GRP ) \times 0.00000297345 + Norm\_read \_count ( HOXA 10 ) \times 0.000234721 - Copy \_ number \_ variation ( MYC ) \times 0.0537056$$

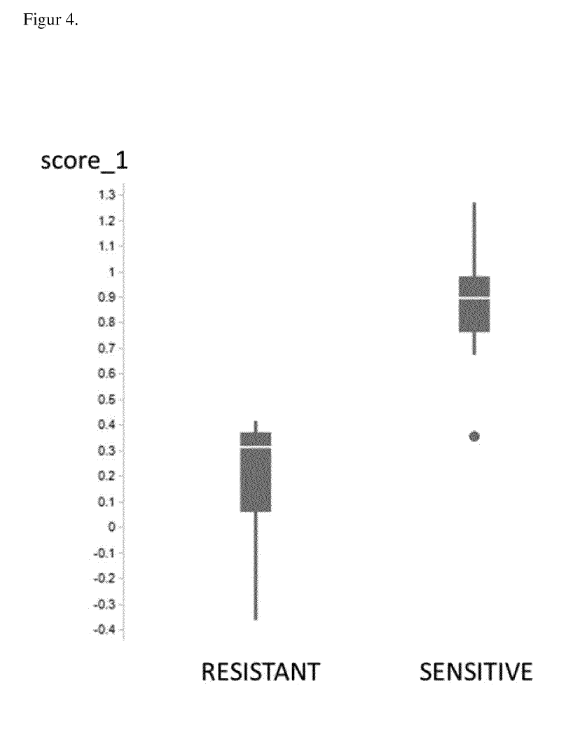

A Signature Score 1>0.5 predicts response to an LSD1 inhibition therapy (Fisher's exact test two-tailed p 0.0001, sensitivity 90%, specificity 100%) as depicted in FIG. 4.

Alternatively, a score was generated from the following equation, obtained by partial least square analysis using the first principal component:

$$Signature Score 2 = 0.483918 + Norm\_read \_count ( ASCL 1 ) \times 0.00000188066 + Norm\_read \_count ( DDC ) \times 0.00000188066 + Norm\_read \_count ( GRP ) \times 0.00000352033 - Copy\_number \_ variation ( MYC ) \times 0.0407898$$

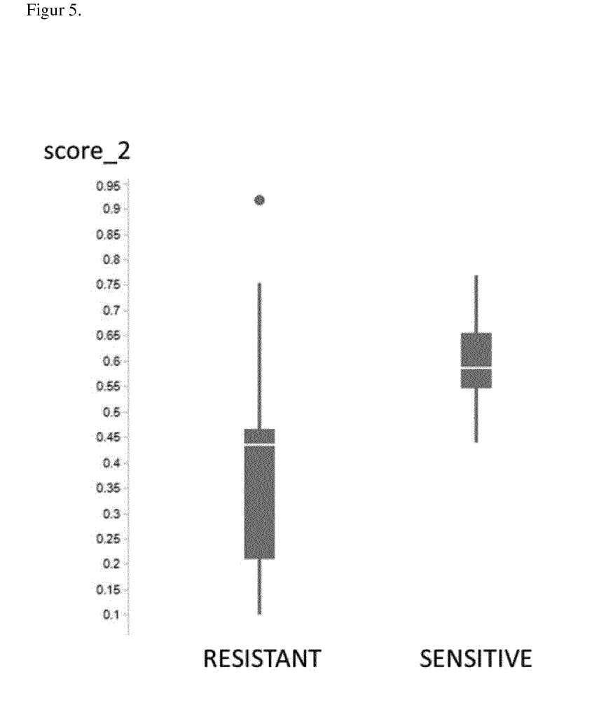

A Signature Score 2>0.5 predicts response to an LSD1 inhibition therapy (Fisher's exact test two-tailed p 0.0055, sensitivity 90%, specificity 77.8%) as depicted in FIG. 5.

Further, a score was generated from the following equation, obtained by partial least square analysis using the first principal component:

$$Signature Score 3 = 0.393569 + Norm\_read \_count ( ASCL 1 ) \times 0.00000182731 + Norm\_read \_count ( DDC ) \times 0.00000189664 + Norm\_read \_count ( GRP ) \times 0.00000342046$$

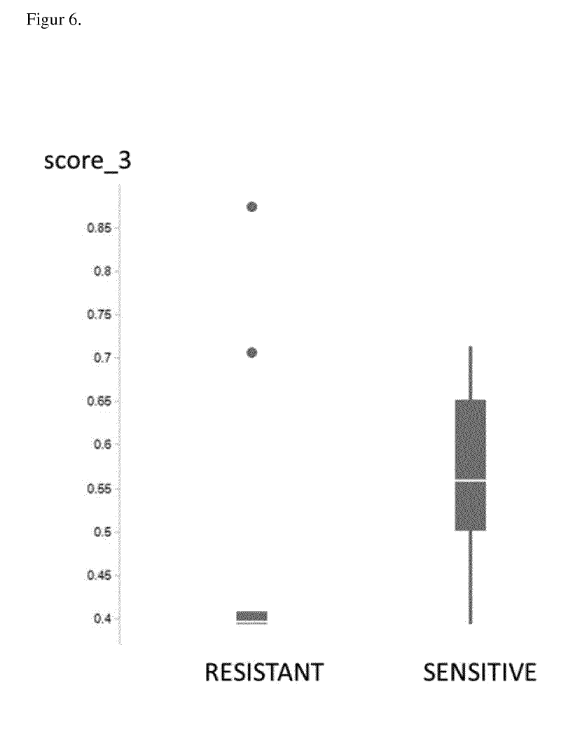

A Signature Score 3>0.45 predicts response to an LSD1 inhibition therapy (Fisher's exact test two-tailed p 0.0055, sensitivity 90%, specificity 77.8%) as depicted in FIG. 6.

A signature score above the reference level indicates a high likelihood of response to treatment with an LSD1 inhibitor, whereas a signature score below said level indicates a lower likelihood to respond to such treatment. A higher score is associated with higher mRNA expression of ASCL1, DDC, GRP, HOXA10, and with lower copy number variations in MYC.

### Example 5. In Vivo Tumor Growth Inhibition

NCI-H510A Models:

7-8-week old athymic nude mice animals were injected with 5×10⁶ H510A cells resuspended in 100 μL of 1:1 mixture of Matrigel® matrix (Corning Inc., Tewksbury/MA, C. S. Hughes et al., Proteomics (2010) 10(9):1886-90) and PBS. Tumors were staged at 200-300 mm³ animals and distributed into dosing groups. (trans)-N1-((1R,2S)-2-phenylcyclopropyl)cyclohexane-1,4-diamine was administered at a dose of 40 μg per kg (upk) five days on/two days off until end of study. Tumor volume was measure biweekly using a digital caliber. The study was concluded when mean tumor volume within control group reached 2000 mm³ or 28 days post-staging. Statistical analysis was performed using unpaired t-test.

NCI-H526 and SHP-77 Models:

8-12-week old nu/nu mice were injected with 1×10⁷ H526 cells or 5×10⁶ SHP-77 resuspended in 100 μL of 1:1 mixture of Matrigel® and PBS. Tumors were staged at 100-150 mm³ animals and distributed into dosing groups. (trans)-N1-((1R,2S)-2-phenylcyclopropyl)cyclohexane-1,4-diamine was administered at a dose of 40 upk five days on/two days off until end of study. Tumor volume was measure biweekly using a digital caliber. The study was concluded when mean tumor volume within control group reached 2000 mm³ or 28 days post-staging. Statistical analysis was performed using unpaired t-test.

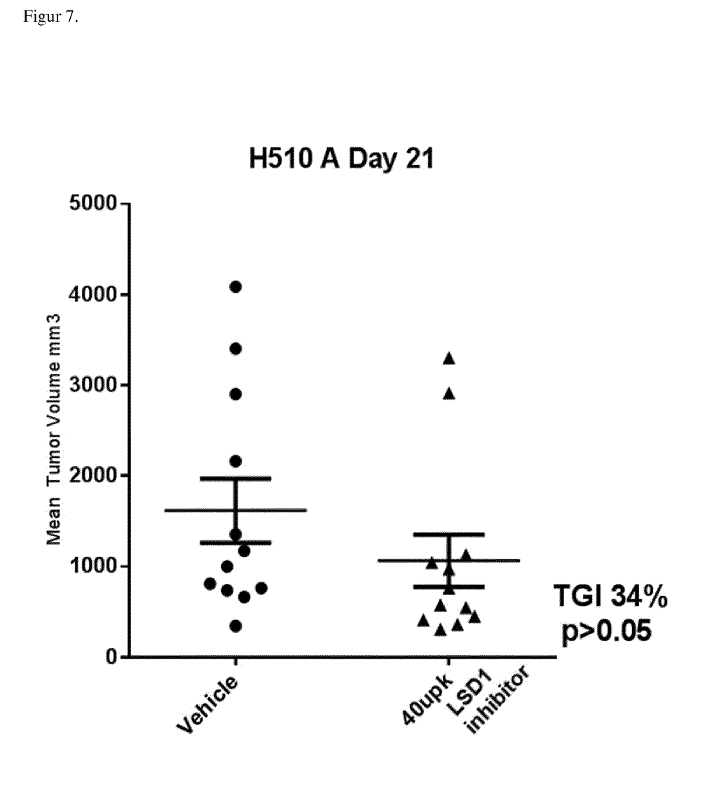

The in vitro activity of the LSD1 inhibitor (trans)-N1-((1R,2S)-2-phenylcyclopropyl)cyclohexane-1,4-diamine translated into in vivo growth inhibition in the H510A xenograft model as shown in FIG. 7. Treatment of (trans)-N1-((1R,2S)-2-phenylcyclopropyl)cyclohexane-1,4-diamine in the “responsive signature” positive cell line H510A model resulted in a modest but measurable tumor growth inhibition of 34% compared to untreated controls after 21 days of dosing. These results suggest that the gene response signature as previously defined may predict in vivo sensitivity to (trans)-N1-((1R,2S)-2-phenylcyclopropyl)cyclohexane-1,4-diamine. The in vivo activity of (trans)-N1-((1R,2S)-2-phenylcyclopropyl)cyclohexane-1,4-diamine has also been assessed in the “response signature positive” SHP-77 and “response signature negative” H526 xenografts to validate the predictability of the gene signature from in vitro results.

1. An in vitro method of identifying a patient having a neoplastic disease as likely to respond to a therapy comprising an LSD1 inhibitor, the method comprising
a) measuring in a sample from the patient the levels of a gene panel, wherein the gene panel comprises one or more genes selected from responder genes and non-responder genes,
b) comparing the levels of the gene panel measured in a) to a reference level,
c) identifying the patient as more likely to respond to the therapy comprising an LSD1 inhibitor when the levels of the responder genes of the gene panel measured in a) in the sample from the patient are above the reference level, and/or when the levels of the non-responder genes of the gene panel measured in a) in the sample from the patient are below the reference level.

2. An in vitro method of identifying a patient having a neoplastic disease as likely to respond to a therapy comprising an LSD1 inhibitor, the method comprising
a) measuring in a sample from the patient the levels of a gene panel, wherein the gene panel comprises one or more genes selected from responder genes and non-responder genes,
b) comparing the levels of the gene panel measured in a) to a reference level,
c) identifying the patient as more likely to respond to the therapy comprising an LSD1 inhibitor when the levels of the responder genes of the gene panel measured in a) in the sample from the patient are above the reference level, and/or when the levels of the non-responder genes of the gene panel measured in a) in the sample from the patient are below the reference level, and
d) administering an effective amount of LSD1 inhibitor.

3. An in vitro method of monitoring efficacy of therapy comprising an LSD1 inhibitor in patient having a neoplastic disease, the method comprising
a) measuring in a sample from the patient prior to start of the therapy the levels of a gene panel, wherein the gene panel comprises one or more genes selected from responder genes and non-responder genes,
b) using the levels of the gene panel measured in a) to calculate the patient's signature score prior to start of the therapy,
c) measuring in a sample from the patient after start of the therapy the levels of the gene panel,
d) using the levels of the gene panel measured in c) to calculate the patient's signature score after start of the therapy,
e) comparing the patient's signature score obtained in d) after start of the therapy with the signature score obtained in b) prior to start of the therapy, and
f) identifying the patient as responding to the therapy when the signature score obtained in d) after start of the therapy are higher than the signature score obtained in b) prior to start of the therapy.

4. A method of treating a patient having a neoplastic disease, the method comprising
a) measuring in a sample from the patient the levels of a gene panel, wherein the gene panel comprises one or more genes selected from responder genes and non-responder genes,
b) comparing the levels of the gene panel measured in a) to a reference level,
c) identifying the patient as more likely to respond to the therapy comprising an LSD1 inhibitor when the levels of the responder genes of the gene panel measured in a) in the sample from the patient are above the reference level, and/or when the levels of the non-responder genes of the gene panel measured in a) in the sample from the patient are below the reference level, and
d) administering an effective amount of LSD1 inhibitor to the patient if likely to respond thereby treating the neoplastic disease.

5. An LSD1 inhibitor for use in treating a patient having a neoplastic disease, wherein the patient is treated if the levels of the responder genes of a gene panel measured in a sample from the patient are above the reference level, and/or when the levels of the non-responder genes of a gene panel measured in a sample from the patient are below the reference level thereby treating the neoplastic disease.

6. An in vitro use of gene panel comprising one or more genes selected from responder genes and non-responder genes for assessing a therapy comprising an LSD1 inhibitor in a patient having a neoplastic disease, wherein levels of the responder genes above a reference level, and/or levels of the non-responder genes below a reference level indicate that the patient should be treated with an effective amount of an LSD1 inhibitor.

7. An in vitro use of a gene panel comprising one or more genes selected from responder genes and non-responder genes for identifying a patient having a neoplastic disease as likely to respond to a therapy comprising an LSD1 inhibitor, wherein levels of the responder genes above a reference level, and/or levels of the non-responder genes below a reference level indicate that the patient is more likely to respond to the therapy.

8. Use of a gene panel comprising one or more genes selected from responder genes and non-responder genes for the manufacture of a diagnostic for assessing a neoplastic disease.

9. Use of a gene panel comprising one or more genes selected from responder genes and non-responder genes for the manufacture of a diagnostic for assessing a therapy comprising an LSD1 inhibitor in a patient having a neoplastic disease.

10. Use of a gene panel comprising one or more genes selected from responder genes and non-responder genes for the manufacture of a diagnostic for assessing the likelihood of response of a patient having a neoplastic disease to a therapy comprising an LSD1 inhibitor.

11. A kit for predicting the likelihood of response to a therapy comprising an LSD1 inhibitor comprising
a) one or more reagents for measuring the levels of a gene panel in a sample, wherein the gene panel comprises one or more genes selected from responder genes and non-responder genes prior to start of the therapy,
b) one or more comparator molecules comprising one or more standard values to which the levels of a gene panel in the sample are compared.

12. The method according to any of claims 1 to 4, the LSD1 inhibitor of claim 5, the use according to any of claims 6 to 10, or the kit of claim 11, wherein the levels measured are mRNA expression levels.

13. The method according to any of claims 1 to 4, the LSD1 inhibitor of claim 5, the use according to any of claims 6 to 10, or the kit of claim 11, wherein the levels measured are mRNA expression levels derived from RNA-sequencing, RT-qPCR or microarrays.

14. The method according to any of claims 1 to 4, 12 and 13, the LSD1 inhibitor according to any of claims 5, 12 and 13, the use according to any of claims 6 to 10, 12 and 13, or the kit according to any of claims 11, 12 and 13, wherein the gene panel comprises one or more genes selected from the group of ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB, BCL2 and MYC.

15. The method according to any of claims 1 to 4, 12 and 13, the LSD1 inhibitor according to any of claims 5, 12 and 13, the use according to any of claims 6 to 10, 12 and 13, or the kit according to any of claims 11, 12 and 13, wherein the gene panel comprises one or more genes selected from the group of MYC, ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2 and HOXA10.

16. The method according to any of claims 1 to 4 and 12 to 14, the LSD1 inhibitor according to any of claims 5 and 12 to 14, the use according to any of claims 6 to 10 and 12 to 14, or the kit according to any of claims 11 to 14, wherein the gene panel comprises one or more genes selected from the group of ASCL1, MYC, HOXA10, DDC, GRP, NCAM1, NCAM2, NEUROD1, KRT8, ENO2, AVP, OXT, SYP, CHGA, CHGB, SOX21 and BCL2.

17. The method according to any of claims 1 to 4, 12 to 14 and 16, the LSD1 inhibitor according to any of claims 5, 12 to 14 and 16, the use according to any of claims 6 to 10 and 12 to 16, or the kit according to any of claims 11 to 14 and 16, wherein the gene panel comprises one or more genes selected from the group of ASCL1, MYC, HOXA10, DDC, GRP, NCAM1, NCAM2, NEUROD1, SOX21 and BCL2.

18. The method according to any of claims 1 to 4 and 12 to 17, the LSD1 inhibitor according to any of claims 5 and 12 to 17, the use according to any of claims 6 to 10 and 12 to 17, or the kit according to any of claims 11 to 17, wherein the gene panel comprises one or more genes selected from the group of ASCL1, MYC, HOXA10, DDC and GRP.

19. The method according to any of claims 1 to 4 and 12 to 18, the LSD1 inhibitor according to any of claims 5 and 12 to 18, the use according to any of claims 6 to 10 and 12 to 18, or the kit according to any of claims 11 to 18, wherein the gene panel comprises one or more genes selected from the group of ASCL1, MYC and HOXA10.

20. The method according to any of claims 1 to 4 and 12 to 19, the LSD1 inhibitor according to any of claims 5 and 12 to 19, the use according to any of claims 6 to 10 and 12 to 19, or the kit according to any of claims 11 to 19, wherein the gene panel consists of one, two, three, four or five genes.

21. The method according to any of claims 1 to 4 and 12 to 20, the LSD1 inhibitor according to any of claims 5 and 12 to 20, the use according to any of claims 6 to 10 and 12 to 20, or the kit according to any of claims 11 to 20, wherein the gene panel consists of two, three or four genes.

22. The method according to any of claims 1 to 4 and 12 to 14 and 16 to 21, the LSD1 inhibitor according to any of claims 5, 12 to 14 and 16 to 21, the use according to any of claims 6 to 10, 12 to 14 and 16 to 21, or the kit according to any of claims 11 to 14 and 16 to 21, wherein the responder genes are selected from the group of ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2, HOXA10, NCAM1, NCAM2, NEUROD1, KRT8, ENO2, AVP, OXT, SYP, CHGA, CHGB and BCL2.

23. The method according to any of claims 1 to 4 and 12 to 22, the LSD1 inhibitor according to any of claims 5 and 12 to 22, the use according to any of claims 6 to 10 and 12 to 22, or the kit according to any of claims 11 to 22, wherein the responder genes are selected from the group of ASCL1, DDC, CEACAM6, LRRIQ4, NR0B2, GRP, CEACAM5, SOX21, OR51E2, SEC11C, BAALC, CCDC40, RAB3B, RGS17, ABCE1, ETS2, CCDC154, SPAG6, PON1, TMEM176A, C1orf127, IGF2BP2, IGFBP5, FAM84A, FOXA2 and HOXA10.

24. The method according to any of claims 1 to 4 and 12 to 14 and 16 to 21, the LSD1 inhibitor according to any of claims 5, 12 to 14 and 16 to 21, the use according to any of claims 6 to 10, 12 to 14 and 16 to 21, or the kit according to any of claims 11 to 14 and 16 to 21, wherein the responder genes are selected from the group of ASCL1, HOXA10, DDC, GRP, NCAM1, NCAM2, NEUROD1, KRT8, ENO2, AVP, OXT, SYP, CHGA, CHGB, SOX21 and BCL2.

25. The method according to any of claims 1 to 4 and 12 to 24, the LSD1 inhibitor according to any of claims 5 and 12 to 24, the use according to any of claims 6 to 10 and 12 to 24, or the kit according to any of claims 11 to 24, wherein the non-responder genes are selected from MYC.

26. The method according to any of claims 1 to 4 and 12 to 25, the LSD1 inhibitor according to any of claims 5 and 12 to 25, the use according to any of claims 6 to 10 and 12 to 25, or the kit according to any of claims 11 to 25, wherein the LSD1 inhibitor is selected from the list of:
4-[[4-[[[(1R,2S)-2-phenylcyclopropyl]amino]methyl]-1-piperidinyl]methyl]-benzoic acid,
(trans)-N1-((1R,2S)-2-phenylcyclopropyl)cyclohexane-1,4-diamine,
(R)-1-(4-(((trans)-2-phenylcyclopropyl)amino)cyclohexyl)pyrrolidin-3-amine,
4-(aminomethyl)-N-((trans)-2-phenylcyclopropyl)cyclohexanamine,
N1-((trans)-2-phenylcyclopropyl)cyclohexane-1,3-diamine,
N1-((trans)-2-phenylcyclopropyl)cyclobutane-1,3-diamine,
N1-((trans)-2-phenylcyclopropyl)-2,3-dihydro-1H-indene-1,3-diamine,
N1-methyl-N4-((trans)-2-phenylcyclopropyl)cyclohexane-1,4-diamine,
N1-((trans)-2-(4-bromophenyl)cyclopropyl)cyclohexane-1,4-diamine,
N1-(2-(o-tolyl)cyclopropyl)cyclohexane-1,4-diamine,
N1-(2-(4-methoxyphenyl)cyclopropyl)cyclohexane-1,4-diamine,
N1-(2-(2-fluorophenyl)cyclopropyl)cyclohexane-1,4-diamine,
N1-(2-(naphthalen-2-yl)cyclopropyl)cyclohexane-1,4-diamine,
N-(4′-((trans)-2-((4-aminocyclohexyl)amino)cyclopropyl)-[1,1′-biphenyl]-3-yl)-2-cyanobenzenesulfonamide,
N1-((trans)-2-(4-(pyridin-3-ylmethoxy)phenyl)cyclopropyl)cyclohexane-1,4-diamine,
and a pharmaceutically acceptable salt thereof.

27. The method according to any of claims 1 to 4 and 12 to 26, the LSD1 inhibitor according to any of claims 5 and 12 to 26, the use according to any of claims 6 to 10 and 12 to 26, or the kit according to any of claims 11 to 26, wherein the LSD1 inhibitor is 4-[[4-[[[(1R,2S)-2-phenylcyclopropyl]amino]methyl]-1-piperidinyl]methyl]-benzoic acid or a pharmaceutically acceptable salt thereof.

28. The method according to any of claims 1 to 4 and 12 to 26, the LSD1 inhibitor according to any of claims 5 and 12 to 26, the use according to any of claims 6 to 10 and 12 to 26, or the kit according to any of claims 11 to 26, wherein the LSD1 inhibitor is (trans)-N1-((1R,2S)-2-phenylcyclopropyl)cyclohexane-1,4-diamine or a pharmaceutically acceptable salt thereof.

29. The method according to any of claims 1 to 4 and 12 to 26, the LSD1 inhibitor according to any of claims 5 and 12 to 26, the use according to any of claims 6 to 10 and 12 to 26, or the kit according to any of claims 11 to 26, wherein the LSD1 inhibitor is (trans)-N1-((1R,2S)-2-phenylcyclopropyl)cyclohexane-1,4-diamine bis-hydrochloride.

30. The method according to any of claims 1 to 4 and 12 to 29, the LSD1 inhibitor according to any of claims 5 and 12 to 29, or the kit of claims 11 and 12 to 29, wherein the sample is taken from a blood specimen, a bone marrow specimen, or a fresh, frozen or formalin-fixed paraffin embedded primary human tumor specimen.

31. The method according to any of claims 1 to 4 and 12 to 30, the LSD1 inhibitor of claims 5 and 12 to 30, the use according to any of claims 6 to 10 and 12 to 30, or the kit according to any of claims 11 to 30, wherein the neoplastic disease is a cancer selected from the group consisting of breast cancer, prostate cancer, cervical cancer, ovarian cancer, gastric cancer, colorectal cancer, pancreatic cancer, liver cancer, brain cancer, neuroendocrine cancer, lung cancer, kidney cancer, hematological malignancies, melanoma and sarcoma.

32. The method according to any of claims 1 to 4 and 12 to 31, the LSD1 inhibitor of claims 5 and 12 to 31, the use according to any of claims 6 to 10 and 12 to 31, or the kit according to any of claims 11 to 31, wherein the neoplastic disease is a blood cancer or lung cancer selected from the group of acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, small cell lung carcinoma (SCLC) and non-small-cell lung carcinoma (NSCLC).

33. The method according to any of claims 1 to 4 and 12 to 32, the LSD1 inhibitor of claims 5 and 12 to 32, the use according to any of claims 6 to 10 and 12 to 32, or the kit according to any of claims 11 to 32, wherein the neoplastic disease is a cancer selected from the group consisting of acute myeloid leukemia (AML), thyroid cancer, melanoma, or small cell lung cancer (SCLC).

34. The method according to any of claims 1 to 4 and 12 to 33, the LSD1 inhibitor of claims 5 and 12 to 33, the use according to any of claims 6 to 10 and 12 to 33, or the kit according to any of claims 11 to 33, wherein the neoplastic disease is small cell lung cancer (SCLC).

35. The invention as hereinbefore described.
